SEQUENCE LISTING (4 sequences)

SEQ ID NO: 1 - protein, 16 aa, Artificial sequence
  Synthetic peptide
LSQETFSDLWKLLPEN

SEQ ID NO: 2 - protein, 29 aa, Homo sapeins
DMRPEIWIAQELRRIGDEFNAYYARRVFL

SEQ ID NO: 3 - protein, 25 aa, Artificial sequence
  Synthetic peptide
WIAQELRRIGDEFNAYYARRKKKKK

SEQ ID NO: 4 - protein, 23 aa, Artificial sequence
  Synthetic peptide
KKKKKMRPEIWIAQELRGDEFNA

(19) United States
(12) Patent Application Publication
(10) Pub. No.: US 2018/0066031 A1
(43) Pub. Date: Mar. 8, 2018

(54) ENZYMATICALLY-CLEAVABLE PEPTIDE AMPHIPHILES

(71) Applicant: The University of Chicago, Chicago, IL (US)

(72) Inventors: Handan Acar, Chicago, IL (US); James LaBelle, Chicago, IL (US); Matthew Tirrell, Chicago, IL (US)

(21) Appl. No.: 15/693,739

(22) Filed: Sep. 1, 2017

Related U.S. Application Data
(60) Provisional application No. 62/383,157, filed on Sep. 2, 2016.

(51) Int. Cl. C07K 14/47

(52) U.S. Cl. CPC C07K 14/4746; A61K 38/00

(57) ABSTRACT

Provided herein are enzymatically-cleavable peptide amphiphiles and methods of use thereof.

### CROSS-REFERENCE TO RELATED APPLICATION

The present application claims priority to U.S. Provisional Application 62/383,157, filed Sep. 2, 2016, which is herein incorporated by reference in its entirety.

### FIELD

Provided herein are enzymatically-cleavable peptide amphiphiles and methods of use thereof.

### BACKGROUND

Recent advances in genomics and computational methods have identified ˜650,000 essential intracellular protein-protein interactions (PPIs) within the human interactome responsible for normal cellular homeostasis of which many also perpetuate malignant transformation (Refs. B1-B2; incorporated by reference in their entireties). One example of such a protein is p53, a tumor suppressor essential for regulating cell stress response through the induction of cell cycle arrest and apoptosis at the level of the mitochondrion (Ref. B3; incorporated by reference in its entirety). Defects in the p53 pathway occur in ˜22 million cancer patients with approximately 50% of these defects due to inactivation of p53 itself and the remaining defects due to aberrancies in other p53 signaling or effector proteins (Refs. B4-B6; incorporated by reference in their entireties). Two of these proteins are MDM2 and MDM4, both of which non-redundantly target p53 for degradation (Refs. B7-B8; incorporated by reference in their entireties). MDM2 is an E3 ubiquitin ligase that targets p53 for ubiquitin-dependent degradation while MDM4 inhibits p53 through PPI-mediated sequestration (Refs. B9-B11; incorporated by reference in their entireties). In many cancers, wild-type TP53's signaling pathway is corrupted by overexpression of these two proteins (Refs. B5, B12; incorporated by reference in their entireties). As a result, there is an urgent need to re-activate p53 particularly in those patients with “complex” p53-pathway copy number alterations who have significantly shorter overall survival when treated with conventional chemotherapies (Refs. B5, B12-B13; incorporated by reference in their entireties).

Small molecule and peptide-based PPI inhibition of p53 with MDM2/4 has been shown to reactivate cell death in vitro and in in preclinical animal models of chemoresistant cancers (Refs. B14-B18; incorporated by reference in their entireties). Leading the way are compounds that, through protein binding mimicry, displace p53 from MDM2 allowing free p53 to reactivate apoptosis. Both hydrocarbon stapled α-helical p53₍₁₄₋₂₉₎ peptides and p53₍₁₄₋₂₉₎ peptide amphiphiles (PAs) are examples of peptide-based therapeutics that inhibit p53:MDM2/4 interactions and have shown pre-clinical promise (Refs. B15, B19-B20; incorporated by reference in their entireties). A hydrocarbon stapled peptide MDM2/4 inhibitor is in clinical trials for advanced solid tumors (Refs. B6, B21; incorporated by reference in their entireties).

### SUMMARY

Provided herein are enzymatically-cleavable peptide amphiphiles and methods of use thereof.

Intracellular delivery of biactive peptides at concentrations necessary for efficacy presents a formidable challenge. Peptide Amphiphiles (PAs) provide a facile method of intracellular delivery and stabilization of bioactive peptides. PAs comprising bioactive peptide headgroups linked to hydrophobic alkyl lipid-like tails prevent peptide hydrolysis and proteolysis in circulation and PA monomers are internalized via endocytosis. However, endocytotic sequestration and steric hindrance from the lipid tail are two major mechanisms that limit PA efficacy for intracellular targets (e.g., intracellular protein-protein interactions (PPIs)). To address these problems, provided herein is a PA platform comprising an enzyme (e.g., cathepsin-B) cleavable linker connecting a bioactive peptide (e.g., a selective p53-based inhibitory peptide) to a lipid tail. The PAs form nanostructures in aqueous solution, and the nanostructures facilitate intracellular delivery and stabilization of bioactive peptides. Once inside a cell, cleavage of the cleavable linker liberates the bioactive peptide allowing for intracellular peptide accumulation and extracellular recycling of the lipid moiety. In some embodiments, a Förster resonance energy transfer (FRET)-based tracking system is used to monitor for cleavage and follow individual PA components in real time. The tracking system may find use in both clinical and research application.

In some embodiments, provided herein are peptide amphiphiles comprising a hydrophobic tail and a bioactive peptide connected by an enzymatically-cleavable linker. In some embodiments, the enzymatically-cleavable linker is cathepsin-B (Cat-B) cleavable. In some embodiments, the bioactive peptide is a therapeutic peptide. In some embodiments, the therapeutic peptide binds to a protein within cells. In some embodiments, the therapeutic peptide mimics (e.g., comprises at least 50% (e.g., 50%, 55%, 60%, 65%, 70%, 75%, 80%, 85%, 90%, 95%, 100%) sequence identity with) a portion of an interaction domain of a target protein (e.g., a domain that facilitates a protein-protein interaction). In some embodiments, the therapeutic peptide binds p53. In some embodiments, the therapeutic peptide comprises at least 50% (e.g., 50%, 55%, 60%, 65%, 70%, 75%, 80%, 85%, 90%, 95%, 100%) sequence identity with SEQ ID NO: 1. In some embodiments, the hydrophobic segment comprises one or more alkyl chains.

In some embodiments, provided herein are compositions comprising a plurality of the peptide amphiphiles described herein self-assembled into a nanostructure (e.g., nanofiber, nanoparticle, etc.) with the hydrophobic tails packed into a core of the nanostructure and the bioactive peptides displayed on the surface. In some embodiments, upon cleavage of the enzymatically-cleavable linkers, the bioactive peptides are released from the nanostructure.

In some embodiments, the peptide amphiphiles described herein comprise an enzymatically-cleavable linker flanked (e.g., one fluorophore on each side) by detectably-distinct fluorophores. In some embodiments, the fluorophore pair is a FRET pair. In some embodiments, upon cleavage of the enzymatically-cleavable linker, a first fluorophore remains attached to the nanostructure and/or hydrophobic tail, and a second fluorophore remains attached to the bioactive peptide. In some embodiments, a FRET signal is produced when the enzymatically-cleavable linker is uncleaved, and the FRET signal is reduced or eliminated when the enzymatically-cleavable linker is cleaved.

In some embodiments, provided herein are compositions comprising a plurality of the FRET-labeled peptide amphiphiles described herein self-assembled into a nanostructure with the hydrophobic tails packed into a core of the nanostructure and the bioactive peptides displayed on the surface. In some embodiments, upon cleavage of the enzymatically-cleavable linker, the functional peptide is released from the nanostructure and FRET between the fluorophores is diminished or eliminated.

In some embodiments, provided herein are methods of delivering a bioactive peptide to an in vivo location, comprising administering a peptide amphiphile and/or nanostructure described herein to a cell, tissue, or subject. In some embodiments, the location of the peptide amphiphile and/or nanostructure is monitored/tracked by fluorescence and/or FRET.

### BRIEF DESCRIPTION OF THE DRAWINGS

FIG. 1. Evaluation and trafficking of enzyme-cleavable peptide amphiphiles (PAs) with FRET compatible fluorophores. To evaluate that capacity of enzyme-cleavable peptide PAs o facilitate cellular penetration and cleavage of peptides from their lipid carriers, in real time, confocal imaging and intracellular FRET analysis were used, with one dye located on the biofunctional peptide and the other on the hydrophobic tail. PAs self-assemble into micelles that are incorporated into the cell through endocytosis. Following endocytosis, cathepsin-B (catB) cleaves the PA through a specific amino acid linker group between the peptide and the hydrophobic tail (blue square). Following cleavage, the peptide and tail are trafficked throughout the cell with the ultimate goal of targeting diseased protein-protein interactions (PPIs) in specific intracellular compartments/organelles depending on the specificity of the peptide being used.

FIG. 2A-C. Chemical structures of (A) cleavable and (B) noncleavable p53₍₁₄₋₂₉₎ peptides on resin with FAM and Tamra fluorophores used for FRET analysis. (C) Recombinant catB was added the side of the chamber and loss of FRET signal is rapidly lost as the enzyme reaches VC-PABA targets (orange) within the focused viewing area of the microscope. No loss of FRET signal was measured against GGG-p53₍₁₄₋₂₉₎ targets (blue) or without the addition of catB (grey).

FIG. 3A-D. Chemical structures of (A) cleavable and (B) noncleavable PAs. Transmission electron microscopy (TEM) images of each PA reveal rounded micelles of similar shape and size (B and D).

FIG. 4A-B. Intracellular accumulation of PAs in HeLa cells. HeLa cells under constant incubation with 10 μM (A) cleavable or (B) noncleavable PAs. FRET incompatible spacing of FAM (labeling p53(14-29)) and Tamra (labeling diC₁₆) allowed for evaluation of PA sequestration over time. Cells incubated with diC₁₆-VC-PABA-p53₍₁₄₋₂₉₎ showed PA accumulation (A) while cells incubated with diC₁₆-GGG-p53₍₁₄₋₂₉₎ did not. Scale bars=20 μm.

FIG. 5A-B. Real-time measurement of catB-mediated cleavage of (A) diC₁₆-VC-PABA-p53₍₁₄₋₂₉₎ and (B) diC₁₆-GGG-p53₍₁₄₋₂₉₎. HeLa cells were incubated for 1 hour with respective PAs and then washed. Location of diC16 and p53₍₁₄₋₂₉₎ were followed for 6 hours. FRET signaling was lower within 2 hours in cells treated with the cleavable diC₁₆-VC-PABA-p53₍₁₄₋₂₉₎ PA and almost undetectable by 6 hours after incubation. Conversely, FRET signaling decreased but still remained in cells treated with noncleavable diC₁₆-GGG-p53₍₁₄₋₂₉₎ PAs. Interestingly, the amount of diC₁₆ and p53₍₁₄₋₂₉₎ in cells treated with diC₁₆-GGG-p53₍₁₄₋₂₉₎ decreased at the same rate as FRET signaling in these cells suggesting trafficking of diC₁₆-GGG-p53₍₁₄₋₂₉₎ out of the cells. Scale bars=20 μm.

FIG. 6A-B. Both diC₁₆-VC-PABA-p53₍₁₄₋₂₉₎ and diC₁₆-GGG-p53₍₁₄₋₂₉₎ are localized in HeLa cells to transferrin-positive early endosomes after 1 hour following incubation (A and B). However, diC₁₆ and p53₍₁₄₋₂₉₎ from diC₁₆-VC-PABA-p53₍₁₄₋₂₉₎ quickly dissociate and can be found in separate locations within the cells (white boxes) with diC₁₆ in transferrin-negative compartments while components from diC₁₆-GGG-p53₍₁₄₋₂₉₎PAs are located in identical locations (yellow boxes). Scale bars=20 μm.

FIG. 7A-B. Superresolution laser scanning confocal microscopy was used to measure the ability of diC₁₆-VC-PABA-p53₍₁₄₋₂₉₎ and diC₁₆-GGG-p53₍₁₄₋₂₉₎ to provide a FRET signal (FRET Efficiency) over time and to quantify the amount of intracellular p53₍₁₄₋₂₉₎ peptide over time. (A) Although the FRET signal decreased in cells incubated with both PAs over time (FIG. 5), remaining diC₁₆-GGG-p53₍₁₄₋₂₉₎ within HeLa cells retained their ability to provide a FRET signal up to 24 hours indicated that the loss of FRET signal in cells treated with diC₁₆-VC-PABA-p53₍₁₄₋₂₉₎ was not due to photobleaching during analysis. (B) Superresolution confocal microscopy measured loss of p53₍₁₄₋₂₉₎ peptide in cells incubated with diC₁₆-GGG-p53₍₁₄₋₂₉₎ compared to those incubated with diC₁₆-VC-PABA-p53₍₁₄₋₂₉₎ by 24 hours indicating extracellular loss.

FIG. 8. Extracellular vesicle examination of media from HeLa cells incubated with diC₁₆-VC-PABA-p53₍₁₄₋₂₉₎ or diC₁₆-GGG-p53₍₁₄₋₂₉₎ The number of extracellar vesicles that are filtered by the red channel (p53₍₁₄₋₂₉₎-containing fraction), as measured by red fluorescence, is 4.28× greater from cells incubated with diC₁₆-GGG-p53₍₁₄₋₂₉₎ compared to those incubated with diC₁₆-VC-PABA-p53₍₁₄₋₂₉₎ This indicates that more p53₍₁₄₋₂₉₎ remained intracellular when treated with diC₁₆-VC-PABA-p53₍₁₄₋₂₉₎ and that intact diC₁₆-GGG-p53₍₁₄₋₂₉₎ PAs were shuttled extracellularly.

FIG. 9. Chemical structure of the enzyme cleavable sequence, (A) Valine-Citrulline-Para(aminobenzoicacid) (VC-PABA)-AMC; (B) Control, non-cleavable sequence, Glycine-Glycine-Glycine (GGG)-AMC; (C) AMC fluorescent intensity change over time following addition of recombinant human cathepsin-B (catB) or PBS control.

FIG. 10. Exemplary BIM BH3 therapeutic PA.

FIGS. 11A-D. Exemplary BIM PA characterization.

FIG. 12. Exemplary BIM PAs bind BCL-2 Proteins.

FIG. 13. Cellular uptake and localization of BIM PAs.

FIG. 14. BIM PA localization.

FIGS. 15A-C. BIM PA-induced cell death and caspase activation.

FIG. 16. BIM PA cell death associated with PARP cleavage, a hallmark of apoptosis.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, the term “peptoid” refers to a class of peptidomimetics where the side chains are functionalized on the nitrogen atom of the peptide backbone rather than to the α-carbon.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, marcomolecules, etc.) and the multicomponent assemblies, complexes, systems, and/or fibers that form as a result.

As used herein, the term “nanostrucuture” refers to macromolecular and/or supramolecular assemblies (e.g., particles (e.g., approximately spherical), filaments (e.g., having a significantly greater length dimension that width or diameter), etc.) with dimensions (e.g., length, width, diameter, etc.) of less than 1 μm (e.g., 900 nm, 800 nm, 700 nm, 600 nm, 500 nm, 400 nm, 300 nm, 200 nm, 150 nm, 100 nm, 75 nm, 50 nm 40 nm, 30 nm, 20 nm, 10, nm, or less, or ranges therebetween). In the case of elongated nanostructures (e.g., nanofibers, nanofilaments, nantotubes, etc.), the length, but not the other dimensions, of the nanostructure may exceed 1 μm.

As used herein, the term “physiological conditions” refers to the range of conditions of temperature, pH and tonicity (or osmolality) normally encountered within tissues in the body of a living human.

As used herein, the term “peptide amphiphile” refers to a molecule that, at a minimum, includes: (i) a non-peptide hydrophobic segment, and (ii) a structural/functional peptide segment. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptidic segment, (2) a structural peptide segment that facilitates interactions between the peptide portions of the peptide amphiphiles upon assembly thereof, and (3) a functional and/or bioactive moiety (e.g., small molecule or peptide).

As used herein, the term “hydrophobic segment” refers to the moiety disposed on one terminus (e.g., N-terminus, C-terminus) of the peptide amphiphile (e.g., an acyl moiety). The hydrophobic segment should be of a sufficient length to provide amphiphilic behavior and micelle (or nanosphere or nanofiber) formation in water or another polar solvent system. Accordingly, in the context of some embodiments described herein, the hydrophobic segment comprises a single, linear acyl chain, for example, of the formula: Cₙ₋₁H₂ₙ₋₁C(O)— where n=6-22. However, other small lipophilic groups may be used in place of the acyl chain. In some embodiments, the packing of the hydrophobic segments of peptide amphiphiles away from the polar solvent (e.g., water) drives assembly of PAs into nanostructures.

As used herein, the term “structural peptide” refers to a portion of a peptide amphiphile, typically disposed adjacent to the hydrophobic segment. The structural peptide, when present, comprises several amino acid residues (e.g., 3-12) selected for their propensity to form hydrogen bonds or other stabilizing interactions (e.g., hydrophobic interactions, van der Waals' interactions, etc.) with the structural segments of adjacent papetide amphiphiles. In some embodiments, nanostructures of peptide amphiphiles form from the packing of hydrophobic moieties at the core of the structure and interactions between the structural peptides facilitating assembly.

As used herein, the terms “functional peptide” or “bioactive peptide” refers to amino acid sequences at the terminus (C-terminus, N-terminus) of the PA opposite the hydrophobic segment. The functional/bioactive peptide mediates the action of sequences, molecules, or supramolecular complexes associated therewith, and carries out the functional (e.g., therapeutic) purpose of the PA. Peptide amphiphiles and structures bearing functional peptides exhibit the functionality of the functional peptide.

As used herein, the term “percent sequence identity” refers to the degree (e.g., 50%, 60%, 70%, 80%, 90%, 95%, 98%, 99%, 100%, ranges therebetween, etc.) to which two polymer sequences (e.g., peptide, polypeptide, nucleic acid, etc.) have the same sequential composition of monomer subunits. If two polymers have identical sequences (e.g., 100% sequence identity) they may be referred to herein as having “sequence identity.” The term “percent sequence similarity” refers to the degree (e.g., 50%, 60%, 70%, 80%, 90%, 95%, 98%, 99%, 100%, ranges therebetween, etc.) with which two polymer sequences (e.g., peptide, polypeptide, nucleic acid, etc.) have similar polymer sequences (e.g., only conservative substitutions between the sequences). For example, similar amino acids are those that share the same biophysical characteristics and can be grouped into the families (see “conservative amino acid substitution” below). If two polymers have sequences that have monomers at each position that share the same biophysical characteristics they may be referred to herein as having “sequence similarity.” The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class; whereas conservative substitutions may involve the exchange of a member of one of these classes for another member of that same class.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y % sequence identity with SEQ ID NO:Z” may have up to X substitutions relative to SEQ ID NO:Z, and may therefore also be expressed as “having X or fewer substitutions relative to SEQ ID NO:Z.”

As used herein, the term “pharmaceutically acceptable carrier” refers to non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., PA nanostructures) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions herein) to a subject or in vivo, in vitro, or ex vivo cells, tissues, and organs. Exemplary routes of administration to the human body can be through the eyes (e.g., intraocularly, intravitrealy, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “fluorophore” refers to a chemical group that may be excited by light to emit fluorescence or phosphorescence. A “quencher” is an agent that is capable of quenching a fluorescent signal from a fluorescent donor. A first fluorophore may emit a fluorescent signal that excites a second fluorophore. A first fluorophore may emit a signal that is quenched by a quencher.

### DETAILED DESCRIPTION

Provided herein are enzymatically-cleavable peptide amphiphiles and methods of use thereof.

Although focus for inhibition of PPIs has classically centered on using small molecules, small molecules are best at targeting PPIs with defined “hot spot” binding residues or concentrated binding foci and often fail to target PPIs with large, diffuse interfaces (>800 Å²) where binding is the summation of geographically distinct low-affinity interactions (Refs., B1, B22; incorporated by reference in their entireties). However, peptides are highly desirable choices to target PPIs due to their fidelity of orthostatic contact points between binding partners (Refs. B23-B24; incorporated by reference in their entireties). Obstacles with peptide-based therapeutics compared to small molecule therapeutics include lower metabolic stability, endosomal entrapment, and cell membrane impermeability (Ref. B14; incorporated by reference in their entireties). PAs represent one strategy to increase cellular impermeability and serum stability of biofunctional peptides. PAs comprise a peptide headgroup linked to a hydrophobic alkyl lipid-like tail that self-assemble into molecules with distinct hydrophobic and hydrophilic ends, akin to natural lipids structurally similar to those within the cellular membrane (Refs. B25-B26; incorporated by reference in their entireties). PAs self-assemble into micellar structures in aqueous medium where the hydrophobic tails are buried within the core while the peptide headgroups remain on the periphery (Refs. 26-27; incorporated by reference in their entireties). PAs also stabilize peptide secondary structure, protect peptides from proteolytic degradation, and delay plasma clearance because of their nanoscale size and shape, while simultaneously enhancing intracellular internalization (Refs, B20, B28-B29; incorporated by reference in their entireties). Examples of pre-clinical PAs are found in diverse areas including tissue targeting, diagnostic imaging, and cancer therapy (Ref. B30; incorporated by reference in its entirety).

Despite the advantages to micellar PA-based systems, one barrier to using PAs for intracellular PPI disruption is endosomal sequestration (Refs. B29, B31; incorporated by reference in their entireties). Most endosomal vesicles recycle back to the cell surface quickly in the early state. Some however become long-lived perinuclear late endo/lysosomal compartments within 30-60 minutes following internalization where peptides have been shown to survive for up to 24 hours (Refs. B20, B30, B32; incorporated by reference in their entireties). As a result, the bulky hydrophobic tail, which is advantageous outside of the cell and during cellular internalization, becomes a membrane “anchor” within the endosome. Thus, enhancing endosomal escape is critical for meaningful clinical transition of PA-based intracellular peptide-based PPI targeting agents.

Endo/lysosomes degrade their contents using amino acid sequence-specific proteases, such as cathepsins, that are activated in low pH. Cathepsin cleavage sequences have been extensively studied as linkers for antibody-drug conjugates with cathepsin-B (catB)-specific sequences being the most commonly used (Ref. B33; incorporated by reference in its entirety). CatB is rarely found in the extracellular matrix, and therefore conjugates produced with catB cleavable linkers remain remarkably stable in circulation (Ref. B34; incorporated by reference in its entirety). Valine-citrulline-PABC (para-amino benzyl carbamate) has been used as an effective endosomally responsive cleavable sequence (and spacer) for anti-cancer prodrugs and antibody-based drug conjugates (Refs., B35-B37; incorporated by reference in their entireties). CatB cleavage occurs C-terminally to the valine-citrulline dipeptide linker while the PABC spacer allows for improved enzyme binding and kinetics, and due to its strong aromatic ring 1,6-elimination ultimately self-immolates following cleavage (Refs. B38-B39; incorporated by reference in their entireties). Given its excellent stability in human plasma, robust cleavage after endocytosis, and potent antigen-specific cytotoxicity experiments were conducted during development of embodiments herein to use a similar strategy to develop PA-based therapeutics.

As such, provided herein are peptide (drug) delivery platforms which allow for peptide dissociation from the carrier. The platform comprises self-assembled nanoparticles of peptide amphiphiles (PAs), carries therapeutic peptides into cells and allows for enzymatic-cleavage-enabled release of the (therapeutic) peptide component from the amphiphilic carrier. In some embodiments, the platform provides tracking and localization of the individual hydrophilic (e.g., therapeutic) and hydrophobic (e.g., structural) components in real time, as well as monitoring release of the peptide from the carrier. In addition to therapeutic uses, the platform also finds use in research and diagnostic applications as well (e.g., elucidation of nanoparticle trafficking, diagnostic, identification of intracellular protein:protein interactions, measurement of self-assembly and dissociation of nanoparticle components, etc.).

Peptide Amphiphiles (PAs) provide a facile method of intracellular delivery and stabilization of bioactive peptides. PAs comprising biofunctional peptide headgroups linked to hydrophobic alkyl lipid-like tails prevent peptide hydrolysis and proteolysis in circulation and PA monomers are internalized via endocytosis. However, endocytotic sequestration and steric hindrance from the lipid tail are two major mechanisms that limit PA efficacy to target intracellular PPIs. To address these problems a PA platform is provided comprising cathepsin-B cleavable PAs where a selective peptide (e.g., p53-based inhibitory peptide) is cleaved from its lipid tail within endosomes allowing for intracellular peptide accumulation and extracellular recycling of the lipid moiety. In experiments conducted during development of embodiments herein cleavage of the PAs was monitored and the individual PA components were followed in real time using a Førster resonance energy transfer (FRET)-based tracking system.

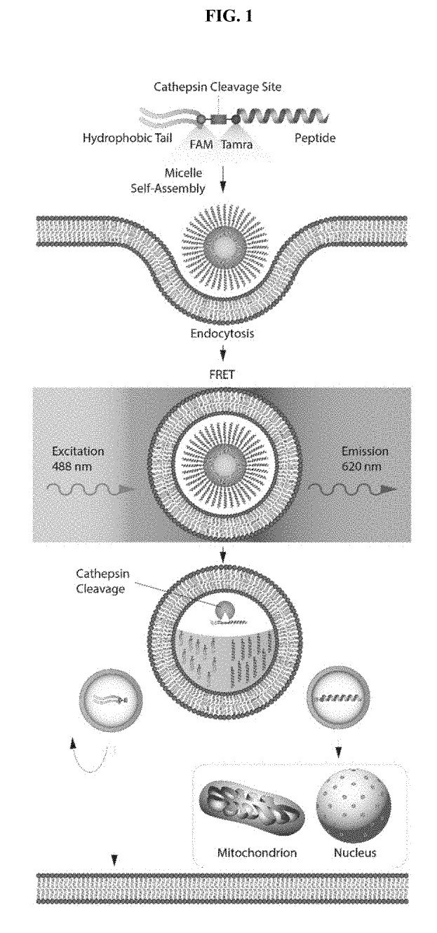

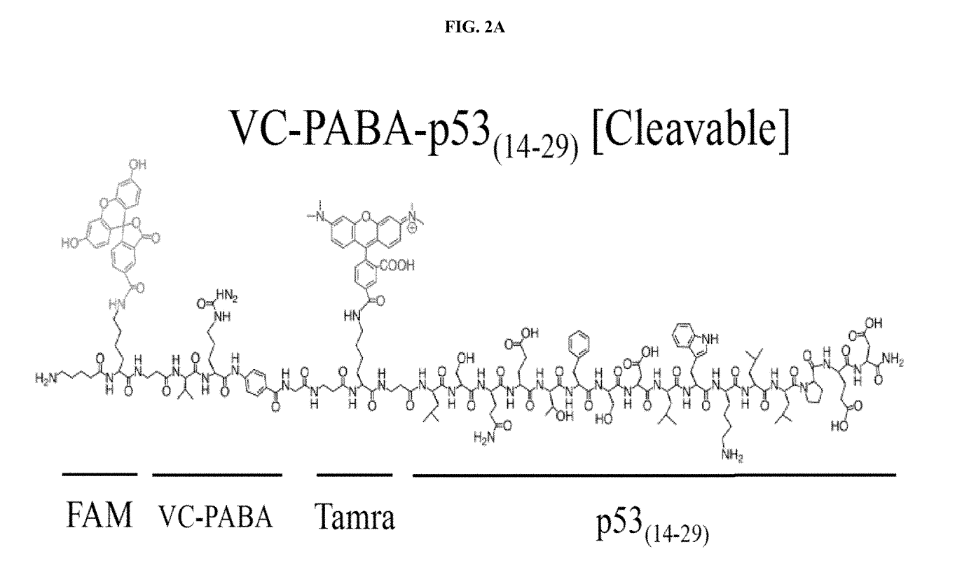

Experiments conducted during development of embodiments herein used p53-based therapeutic peptide (p53₍₁₄₋₂₉₎) PAs prepared with double palmitic acid (diC₁₆) hydrophobic tail and valine-citrulline-PABA (para-amino benzoic acid) (VC-PABA) synthesized between the peptide and the hydrophobic tail to allow for intracellular transport and peptide accumulation. The p53₍₁₄₋₂₉₎ peptide and diC₁₆ were coupled with Förster resonance energy transfer (FRET) compatible chromophores to monitor intracellular PA cleavage in real-time. The experiments conducted during development of embodiments herein were able to individually track diC₁₆ tails and p53₍₁₄₋₂₉₎ peptides using these fluorophores to gain a better understanding of PA cellular internalization, peptide accumulation, lipid tail-mediated membrane sequestration, and intact PA intracellular/extracellular movements that can be extended to other PA-based systems (FIG. 1).

In some embodiments, the platform PAs comprises a hydrophobic tail, enzymatically-cleavable linker segment, and a functional (hydrophilic) peptide. In some embodiments, the linking of the hydrophobic tail and hydrophilic peptide results in the formation of nanostructures of PAs (e.g., micelles) displaying their functional peptides on the exterior. Upon cleavage of the enzymatically-cleavable linker segment (e.g., by an enzyme (e.g., cathepsin-B) at a target site (e.g., a cancer cell, etc.), the functional (e.g., therapeutic) peptide is release to more efficiently exert its (therapeutic) function.

In particular embodiments, enzymatically-cleavable linker segment (which connects the hydrophobic tail and the functional peptide) is flanked by fluorophores (e.g., a FRET pair). In some embodiments, FRET occurs between the fluorophores when the PA is uncleaved. However, upon cleavage of the enzymatically-cleavable linker segment, the functional (therapeutic) peptide and its fluorophore are released from the PA nanostructure and its fluorophore, thereby exceeding the FRET distance and reducing or eliminating the FRET signal. Such a configuration allows for monitoring the location of the PA nanostructures (e.g., within a cell, tissue subject, etc.) and the release of the functional peptide from the carrier. In experiments conducted during development of embodiments herein, using an exemplary FRET PA system, individual peptides and hydrophobic tails were tracked intracellularly in relation to endosomes. The localization of the cleaved peptide and hydrophobic tail within various intracellular compartments was observed, at times located together and at times dissociated from one another.

In some embodiments, the enzyme(s) that intracellularly cleave the peptide from the lipophilic carrier are cathepsins (e.g., cathepsin-B). Cathespins are endosomal proteases present in all mammalian cells. Some cathepsin isoforms can also be extracellarly secreted, especially from malignant cells. The normal digesting mechanism of endo-lysosomes triggers the secretion and activation of these proteases within the acidic endosomal environment. The specific amino-acid sequences cleaved by cathepsin-B have been studied and used in antibody-drug combinations (refs. A1-A6; incorporated by reference in their entireties). Unlike other cathepsins, cathepsin-B is rarely found in the extracellular matrix. Therefore, cathepsin-B-cleavable PAs (e.g., comprising a cathepsin-B-specific cleavable sequence), are stable in circulation until they reach the desired intracellular location (ref. A5; incorporated by reference in its entirety).

In particular embodiments, the PAs herein comprise a valine-citrulline-PABC (Val-Cit-PABC) sequence which is efficiently cleaved by cathepsin-B (Refs. A5, A7; incorporated by reference in their entireties).

Experiments were conducted during development of embodiments herein to observe the location and timing of intracellular PA dissociation upon cleavage and determine the fate of the functional peptide and the hydrophobic tail before and after cleavage within the endo-lysosomal vesicle.

In some embodiments, peptide amphiphiles comprise a hydrophobic (non-peptide) segment linked to a peptide. In some embodiments, the peptide comprises a structural segment (e.g., comprising amino acids that interact with the amino acids of adjacent PAs to encourage supramolecular assembly), and/or a bioactive segment (e.g., a peptide configured to be exposed on the surface of a supramolecular PA nanostructure).

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the hydrophobic segment.

In some embodiments, the hydrophobic segment is incorporated at the N- or C-terminus of the peptide and is composed of a fatty acid or other acid that is linked to the N- or C-terminal amino acid. In aqueous solutions, PA molecules assemble (e.g., into nanostructures) that bury the hydrophobic segment in their core with the peptide segments aligning around the outer surface. In some embodiments, structural peptide segments for a outer surface. In some embodiments, bioactive peptides are displayed upon the surface. The peptide segments interact via intermolecular hydrogen bonding or other supramolecular interactions to facilitate nanostructure formation.

In some embodiments, PAs herein comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment to yield a peptide amphiphile molecule. In some embodiments, a plurality of such PAs will self-assemble in water (or aqueous solution or other polar solvent) into a nanostructure (e.g., nanofiber, nanoparticle, etc.).

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment is a di-acyl moiety. In some embodiments, the hydrophobic segment comprises one or more (e.g., 2) acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprises at least 50% (e.g., 50%, 55%, 60%, 65%, 70%, 75%, 80%, 85%, 90%, 95%, 99%, 100%, or ranges therebetween (e.g., >70%, >80%, >90%, etc.)) sequence identity or similarity (e.g., conservative or semi-conservative) to one or more of the peptide sequences described herein.

In some embodiments, provided herein are peptide amphiphiles comprising a hydrophobic segment and a peptide segment connected by a cleavable linker. The cleavable linker may be a peptide or non-peptide group, and may be cleavable by any suitable mechanism (e.g., photocleavable, chemically cleavable, enzymatically cleavable). In particular embodiments, the cleavable linker is enzyme cleavable (e.g., CatB-cleavable). In some embodiments, the cleavable moiety of the cleavable linker is connected directly to the peptide and/or hydrophobic segments. In some embodiments, the cleavable moiety of the cleavable linker is connected to the peptide and/or hydrophobic segments by one or more spacer moieties. In some embodiments, the cleavable PA platform described herein allows for assembly of PA nanostructures, administration of such nanostructures (e.g., to a cell, subject, etc.), and then release of bioactive peptides from the nanostrcutures upon cleavage of the cleavable linker upon exposure to desired conditions (e.g., contact with a specific enzyme).

In some embodiments, the peptide amphiphiles herein comprise a bioactive peptide. In some embodiments, the bioactive peptide is positioned (e.g., terminally) within the PA molecule to result is display of the bioactive peptide on the exterior of nanostructures of assembled PAs. Embodiments herein are not limited by the identity of the bioactive peptides that may find use herein. In some embodiments, bioactive peptides that find use herein comprise sequences that exert a therapeutic or other biologic activity upon intracellular release.

An exemplary bioactive peptide that finds use in embodiments herein is the p53-inhibitory peptide of SEQ ID NO: 1. In some embodiments, a bioactive peptide for use in embodiments herein comprises one or more (e.g., 1, 2, 3, 4, 5, 6, 7, 8, etc.) substitutions (e.g., conservative, semi-conservative, non-conservative) relative to SEQ ID NO: 1. In some embodiments, a bioactive peptide for use in embodiments herein has at least 50% (e.g., 50%, 55%, 60%, 65%, 70%, 75%, 80%, 85%, 90%, 95%, 99%, 100%, or ranges therebetween (e.g., >70%, >80%, >90%, etc.)) sequence identity and/or sequence similarity with SEQ ID NO: 1. In some embodiments, a peptidomimetic of SEQ ID NO: 1 comprising one or more unnatural amino acids, amino acid analogs, and/or peptoid amino acids is within the scope herein. However, embodiments herein are not limited to PAs comprising peptide segments related to SEQ ID NO: 1. For example, any other p53-inhibitory peptides that are suitable for inclusion in the PA platform described herein may find use in embodiments herein. Further, embodiments herein are not limited to PAs comprising p53-inhibitory peptides. Other peptides that inhibit the growth, spread, metastasis, etc. of cancer and/or tumors may find use in the cleavable PAs described herein. Additionally, the PA platform described herein is not limited to the treatment of cancer. Other therapeutic peptides that find use in the treatment of prevention of disease may find use as the peptide segment of the PAs herein. Other peptides that have disrupt an intracellular protein-protein interaction and/or exhibit another desired intracellular functional characteristic may find use as the bioactive peptide in embodiments herein. The PAs herein are also not limited to therapeutic uses. For example, any peptide that a user, clinician, researcher, etc. desires to deliver to a cell, subject, in vitro system, etc. may find use in some embodiments herein. The scope of embodiments herein should not be limited to the specific sequences disclosed herein.

A feature of the PA amphiphiles described herein is the cleavable segment or cleavable linker. In some embodiments, a linker separating the bioactive peptide from the hydrophobic segment is cleavable upon exposure to desired conditions. Such conditions may include electrostatic environment (e.g., high pH, low pH), temperature (e.g., heat cleavable), a select wavelength of light (e.g., photocleavable), a chemical compound (e.g., chemically cleavable), an enzyme (e.g., enzyme cleavable). Most embodiments herein are described in connection with an enzyme-cleavable linker, but other cleavable linkers are within the scope herein. In particular embodiments, the linker is a peptide and/or non-peptide segment that is prone to cleavage by an enzyme. In some embodiments, the linker is cleaved by an enzyme that is native to an intracellular environment. In some embodiments, the enzyme is a lysosomal protease, such as those described in Brix K. Lysosomal Proteases: Revival of the Sleeping Beauty. In: Madame Curie Bioscience Database [Internet]. Austin (Tex.): Landes Bioscience; 2000-2013; incorporated by reference in its entirety. In some embodiments, the enzyme is an aspartic protease, cysteine protease, or serine protease, and the cleavable linker comprises a suitable peptide or non-peptide sequence for cleavage thereby. Examples of suitable enzymes for cleavage of corresponding cleavable linkers include cathepsin A, cathepsin B, cathepsin D, cathepsin H, cathepsin K, cathepsin L, cathepsin S, asparaginyl endopeptidase, etc. In some embodiments, the enzyme is a non-lysosomal protease (e.g., calpain, prolyl/glycyl proteases, etc.). Cleavage site for protease are understood in the field and or readily determined by methods understood by those in the art.

In some embodiments, the linker tethering the bioactive peptide to the hydrophobic segment is a cathepsin B (catB)-cleavable linker. In some embodiments, the catB-cleavable linker comprises a native catB cleavage sequence. In some embodiments, the cleavable linker comprises a sequence described, for example in one of Refs. B35 or B40-B43 (herein incorporated by reference in their entireties). In some embodiments, the enzyme-cleavable linker comprises valine-citrulline (VC), valine-citrulline-p-aminocarbamate (VC-PABC), or valine-citrulline-p-aminobenzoate (VC-PABA).

In some embodiments, one or more fluorophores are included in the PAs described herein to facilitate monitoring/tracking of the PAs, components thereof (e.g., hydrophobic segments, peptide segments, etc.) within a cell, tissue, or subject. A fluorophore may be attached to the PA at any suitable location (e.g., peptide terminus, within the peptide, between the peptide and cleavable linker, within the cleavable linker, between cleavable linker and hydrophobic segment, within the hydrophobic segment, hydrophobic terminus).

In some embodiments, a single fluorophore is attached to (or within) a PA. In some embodiments, the fluorophore allows for monitoring of nanostructure assembly and/or localization of nanostructures within a cell, tissue, or subject.

In some embodiments, a single fluorophore is attached to (or within) the bioactive peptide of a PA. In some embodiments, the fluorophore allows for monitoring of nanostructure assembly, localization of nanostructures within a cell, tissue, or subject, and/or localization of the bioactive peptide (e.g., upon release from the PA/nanostructure) within a cell, tissue, or subject.

In some embodiments, a single fluorophore is attached to (or within) the hydrophobic segment of a PA. In some embodiments, the fluorophore allows for monitoring of nanostructure assembly, localization of nanostructures within a cell, tissue, or subject, and/or localization of the hydrophobic segment (e.g., upon release from the PA/nanostructure) within a cell, tissue, or subject.

In some embodiments, a pair of fluorophores are attached to (or within) a PA. In some embodiments, a first fluorophore is attached to (or within) the bioactive peptide and a second fluorophore is attached to (or within) the hydrophobic segment. In some embodiments, the fluorophores allow for monitoring of nanostructure assembly, localization of nanostructures within a cell, tissue, or subject, localization of the bioactive peptide (e.g., upon release from the PA/nanostructure) within a cell, tissue, or subject, and/or localization of the hydrophobic segment (e.g., upon release from the PA/nanostructure) within a cell, tissue, or subject. In some embodiments, the pair of fluorophores are a FRET pair. In some embodiments, the fluorophores are located on the PA within the Förster distance of each other (e.g., 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 20 nm, or ranges therebetween). In some embodiments, the distance between the FRET pair is selected based on the identity of the fluorophores. In some embodiments, the fluorophores are located immediately adjacent to either side of the cleavable linker. In some embodiments, FRET is measurable/detectable from a FRET pair located within a PA when the PA is intact (e.g., uncleaved), whether or not the PA is within a nanostructure. In some embodiments, upon cleavage of the cleavable linker, the FRET signal is diminished or eliminated.

In some embodiments, a fluorophore and corresponding quencher are located on/within a PA. In such embodiments, emission from the fluorophore is quenched when the PA is intact (e.g., uncleaved), whether or not the PA is within a nanostructure. In some embodiments, upon cleavage of the cleavable linker, the fluorophore is no longer quenched and the fluorescent signal is detected.

Exemplary fluorophores for use in embodiments herein include Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine, 5-(2′-aminoethyl)aminonaphthalene-1-sulfonic acid (EDANS), Alexa dyes, etc.

In some embodiments, a nanostructure comprises many PA or the same molecular structure (e.g., same hydrophobic segment, same cleavable linker, same flurophore(s), same bioactive peptide, etc.). In some embodiments, provided herein are nanostructures of multiple (e.g., 2, 3, 4, 5, 6, or more) distinct PAs. In some embodiments, PAs described herein are combined with PAs that are not, alone, within the scope herein, to yield a PA nanostructure within the scope herein. In some embodiments, PAs described herein are combined with PAs lacking a bioactive peptide (e.g., comprising a structural peptide only), lacking a cleavable linker, etc. In some embodiments, different PAs (e.g., FERET-labeled and unlabeled, comprising different fluorophores (e.g., allowed FRET detection of nanostructure assembly), comprising different bioactive peptide, comprising different hydrophobic segments, comprising different cleavable linkers, etc.) within the scope herein are combined to form a nanostructure.

In some embodiments, methods are provided for treating a disease of condition (e.g., cancer) in a subject comprising administering a nanostructure of the PAs described herein to a subject. In particular embodiments, the PAs target a protein or protein-protein interaction (PPI) within a cell or cell type (e.g., tumor cells) and inhibit a pathway that contributes to cancer, cell proliferation, metastasis, angiogenesis, etc. In particular embodiments, the PAs herein and nanostrcutures thereof find use in the treatment of cancer. Embodiments herein are not limited by the proteins the PA herein may target, the PPIs they inhibit, or the cancers they find use in treating. Non-limiting examples of cancers that may be treated with the PAs, nanostrcutures, and methods described herein include, but are not limited to: cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is a melanoma (e.g., metastatic malignant melanoma), renal cancer (e.g. clear cell carcinoma), prostate cancer (e.g. hormone refractory prostate adenocarcinoma), pancreatic cancer (e.g., adenocarcinoma), breast cancer, colon cancer, gallbladder cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, the cancer is a solid tumor cancer.

### EXPERIMENTAL

The following are descriptions of experiments and the results/data derived therefrom. The experiments conducted during development of embodiments herein provide support for, but do not limit, the full scope of embodiments herein.

### Materials and Methods

### Peptide Amphiphile (PA) Synthesis:

Amino acids were purchased from Protein Technologies Inc. Peptide p53₍₁₄₋₂₉₎ (LSQETFSDLWKLLPEN) was synthesized on Rink amide resin (Novabiochem) using a standard Fmoc solid phase peptide synthesis strategy on an automated peptide synthesizer (Protein Technologies Inc.) (Ref. B51; incorporated by reference in its entirety). Coupling of 5-Carboxyfluorescein (FAM) and 5(6)-Carboxytetramethylrhodamine (Tamra) (Novabiochem) were performed through the orthogonal side chain protections of Fmoc-Lys(Mtt)-OH and Fmoc-Lys(Dde)-OH, (Novabiochem), respectively. 2× eq (with respect to resin substitution) of each dye dissolved in dimethylformamide (DMF) with 4×DIPEA and 1.95×HATU were used for coupling to the ε-amine of lysine for 24 hours at room temperature. The di-alkyl lipid acid 4-(1,5-bis(hexadecyloxy)-1,5-dioxopentan-2-ylamino)-4-oxobutanoic (diC₁₆COOH) was synthesized as described in Ref. B65; incorporated by reference in its entirety. The Fmoc group of the N-terminal lysine was cleaved with 20% piperidine in DMF, and the free α-amine group of the lysine-containing peptides were conjugated with 2×diC₁₆COOH hydrophobic tail in DMF with 4×DIPEA and 1.95×HATU (Ref. B66; incorporated by reference in its entirety). The coupling reaction shook for 24 hours at room temperature. Complete cleavage from the resin was achieved using a trifluoroacetic acid (TFA):triisopropylsilane:water (98:1:1) solution. The resulting product was precipitated in cold diethyl ether prior to purification.

Modified peptides were purified by reverse phase preparative high performance liquid chromatography (RP-HPLC, Prominence, Shimadzu Corporation, Kyoto, Japan) with an XBridge Prep C8 OBD column (Waters Corporation, Milford, Mass.) at 50° C. (flow rate: 10 mL/min from 10% to 100% within 55 min). Product identity was confirmed using matrix-assisted laser desorption/ionization (MALDI) mass spectrometry (Bruker Ultraflextreme MALDI-TOF) (Ref. B29; incorporated by reference in its entirety).

### Micelle Formation:

PAs were dissolved in chloroform and the solvent evaporated under N₂ gas to form a layer on the wall of the eppendorf tube. Milli-Q water (or PBS for cell culture experiments) (pH 7.4) was added to the PAs, sonicated for 1 hour at 60° C., and then incubated in a hot bath without sonication for 1 hour for 60° C. After cooling to room temperature the micelle solutions were filtered through a 0.45 μm polycarbonate syringe filter (Millipore).

### Critical Micelle Concentration (CMC):

CMC was performed as described in Ref. B67; incorporated by reference in its entirety. A range of PA concentrations (from 512 μM to 0.01 μM in half increments) were prepared in a 1 μM DPH aqueous solution and equilibrated for 1 hour at room temperature. Solutions were plated in triplicates in a black 96-well plate and their fluorescent intensity was measured using Tecan Infinite M200 PRO plate reader (Mannedorf, Switzerland). Data were fit with two trend lines for low and high intensity measurements and CMC was calculated as the inflection point where the two trend lines meet (Ref. B68; incorporated by reference in its entirety).

### Dynamic Light Scattering (DLS):

Micelle size was assessed using dynamic light scattering (DLS, Brookhaven Instruments, Holtzville, N.Y., USA). Stock solutions of 0.5 mM micelles were prepared in water as described above and DLS measurements were performed at 90° angle and 637 nm system consisting of a BI-200SM goniometer and a BI-9000AT autocorrelator. Hydrodynamic radii were determined via the Stokes-Einstein equation using the diffusion coefficient determined from the auto correlation function.

### Transmission Electron Microscopy (TEM):

Ultrathin carbon type-A 400 mesh copper grids (Ted Pella, Redding, Calif., USA) were loaded with 5 μL of 0.5 mM PA micelles and allowed to dry. Grids were washed with several drops of water and then negatively stained with 1% aqueous phosphotungstic acid for 3 min. The excess solution was then removed and grids were left to dry. Grids were imaged on a FEI Tecnai 12 TEM using an accelerating voltage of 120 kV.

### Synthesis of Peptide-AMC:

Fmoc-Lys(carbamate Wang resin)-AMC (Novabiochem) was used to synthesize the cleavable groups on the resin individually. Basic SPPS, as detailed above, was used to synthesize: VC-PABA-AMC and GGG-AMC peptides. 98% TFA cleavage was used to clave the peptide-AMC from the resin.

### Cathepsin-B Cleavage Testing:

Peptide-AMC: Recombinant human liver cathepsin-B (Sigma Aldrich) was used for the in vitro experiments. 0.25 μM DTT used in 0.25 μM HEPES in PBS (pH 5) as activation buffer. Peptides were dissolved in activation buffer to a final concentration as 1 mM. 0.5 μL of catB enzyme or vehicle control was added into the peptide solution. Plate reader analysis with Tecan Infinite M200 PRO plate reader (Mannedorf, Switzerland) with triplicates of each sample were performed in 96 well plates. The intensity of the excited dye at 388 nm was measured at 440 nm. Dual dye-labeled peptide on resin: CatB and activation buffer were prepared as described above. Dye-labeled peptides were again left on the resin. Resin was washed with methanol, dried in a vacuum overnight, and then immersed in PBS (pH 7.4) for 1 hour at 37° C. PBS was then drained followed by addition of 1 mL activation buffer and 5 μL of catB for 100 mg of resin. Control testing was performed in the same solution without the addition of catB. Supernatant was collected after 3 hours and 24 hours. The florescence intensity of triplicates of each sample was measured with plate reader FAM (λexc=485 nm, λem=535 nm) and Tamra (λexc=520 nm, λem=620 nm).

FRET Measurements with Wide Field Microscope:

Peptide-laden resins were washed with methanol, vacuum dried overnight and then immersed in PBS (pH 7.4) for 1 hour at 37° C. After the PBS was drained, 100 μL of activation buffer and 5 μL of enzyme was added to the edge of the well containing peptide-resin at 37° C. FRET change based on enzyme cleavage was measured by a home-built 2-channel FRET imaging system. The system is based on an inverted microscope (Nikon Ti) with differential interference contrast (DIC) imaging components. The excitation light from a CW-laser source (λₑₓ=488 nm, Cobalt) is combined with a fiber optics and sent to the total internal reflection fluorescence (TIRF) illumination combiner attached to the back port of the microscope. Light was reflected by a dichroic beam splitter (quadband) and focused onto the resin beads attached to the two dye-labeled peptides by a high numerical-aperture oil-immersion objective (1.4 NA, 100×). The fluorescence signal emitted from the FRET donor (FAM) and acceptor (Tamra) was unpolarized and relayed to the camera with combination two 200 mm achormatic doublet lens applying the 4f relay system methods. The emission signals were passed through a 500 nm long pass filter to obtain the fluorescence images and intensity trajectories. A dichroic beam splitter (555 nm long pass) at an orientation of 45° angle on the direction of the signal separates out beam depending on the color of light. A dichroic beam splitter transmits acceptor signal and reflect the donor signal. Donor (FAM) and acceptor (Tamra) signals were passed through band pass filters at 525/50 nm and 605/50 nm respectively. The donor and acceptor channels were then reflected by two mirrors and focused to a 1024×1024 pixel electron multiplying charge coupled device (EMCCD) camera (Andore iXon 888) through a 2-inch achromatic doublet lens. The fluorescence signals were recorded using a time lapse-video with acquisition times of 10 ms and interval times of 30 s or 60 s.

### Cell Culture:

HeLa cells were maintained in DMEM (Invitrogen) supplemented with 10% FBS, 100 U ml⁻¹ penicillin/streptomycin, 2 mM L-glutamine, and 0.1 mM MEM nonessential amino acids. Cells were grown at 37° C., humidified atmosphere and 5% CO₂. Cells were allowed to attach on the surfaces overnight (12 h).

### Peptide Amphiphile Treatment of Cells for Confocal Analysis:

HeLa cells were incubated with 2.5 μM PAs and 0.1 μM transferrin (Alexa Fluor 647 labeled, Thermo Fisher Science) for 1 hour in Advanced DMEM (Invitrogen) supplemented with 1% FBS. The media was then removed and cells were washed and either fixed immediately with 4% paraformaldehyde in PBS for 10 minutes at room temperature, or replaced with new peptide-free media and cells were allowed to incubate for another 1, 2, or 5 hours before being fixed. The fixed cells were then washed and left in PBS before being imaged. For accumulation experiments, 10 μM PAs were incubated individually and left on the cells for 24 h and the confocal images were taken in different time periods from live cells.

### Confocal and Superresolution Microscopy:

Images were taken by Marianas Yokogawa type spinning disk (inverted confocal microscope). The following lasers were used: (1) green: λexc=488 nm, filter: green; (2) Red: λexc=565 nm, filter: red; (3) Transferrin: λexc=640 nm, filter: far red; (4) FRET channel: λexc=488 nm, filter: red. Superresolution images were taken on a Leica SP5 II STED-CW Superresolution Laser Scanning Confocal Microscope. All imaging was performed at the Integrated Light Microscope Core Facility at the University of Chicago. Images were analyzed by ImageJ software.

### Extracellular Vesicle Analysis:

HeLa cells were grown in the T25 with 10% FBS. Cells were washed twice with PBS and incubated with (1) 10 μM diC₁₆-GGG-p53₍₁₄₋₂₉₎, (2) 10 μM diC₁₆-VC-PABA-p53₍₁₄₋₂₉₎, and (3) media alone for 1 hour in Advanced DMEM supplemented with 1% FBS. This media was then removed and cells were washed twice with PBS. New 10% FBS media was then added on the cells. Following incubation for 6 and 24 hours, 1 mL of media was collected from each of the samples and analyzed using a Nanosight NS300 flow cell (Nanosight, UK) following the manufacturer protocol. Nanoscale particles (10-1000 nm) were analyzed using the NTA software for size distribution and total number of particles per frame. Particles were also tracked using red filters to detect red-laden particles. The ratio of detected red particles per mL to total particle per mL for each sample was then calculated.

### Example 1

### CatB-Cleavable Linker Evaluation

A variety of enzyme-cleavable peptide sequences used in antibody-drug and peptide-drug conjugates were tested for efficacy in the systems herein (Ref. B35, B40-B43; incorporated by reference in their entireties). The enzyme-cleavable peptide sequence, valine-citrulline (VC), was selected for subsequent experiments, as it yielded the fastest and most complete cleavage. PABC was substituted for PABA, because PABA has equivalent functional cleavage characteristics in pre-clinical testing and contains a moderately electron withdrawing carboxylic acid group making it more stable during solid phase peptide synthesis (Ref. B44; incorporated by reference in its entirety).

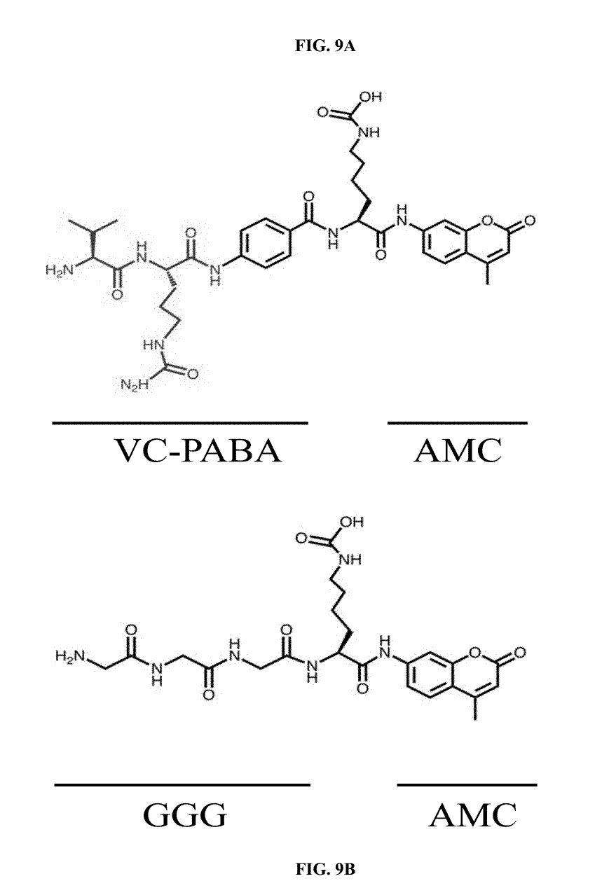

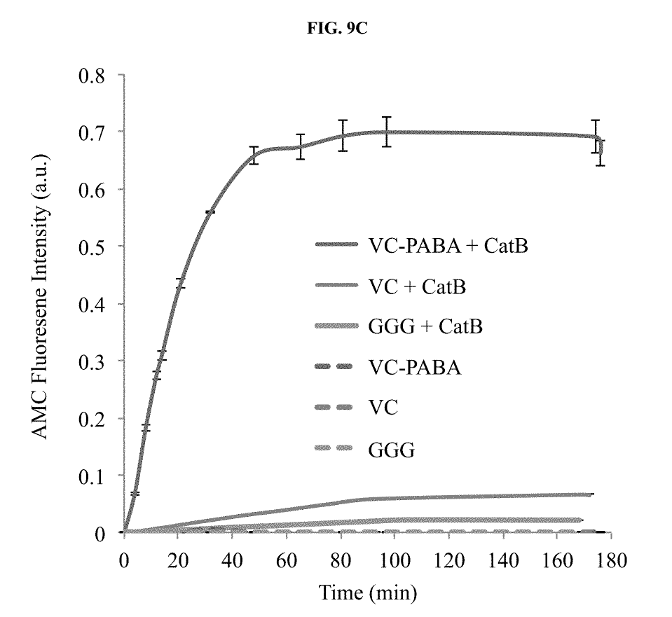

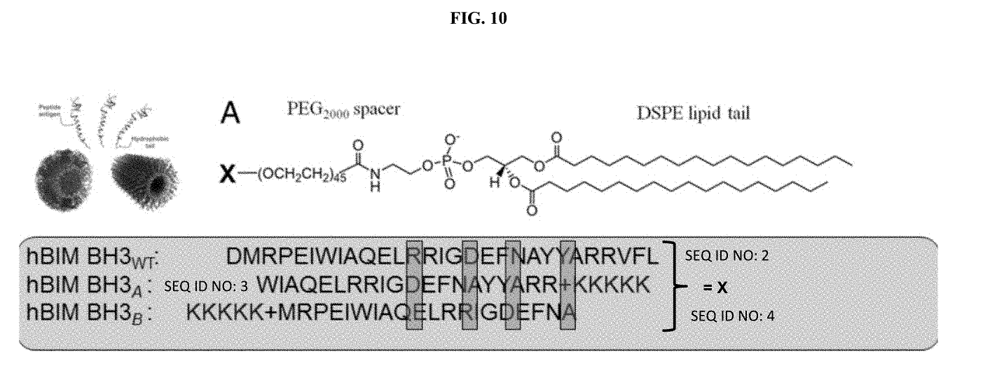

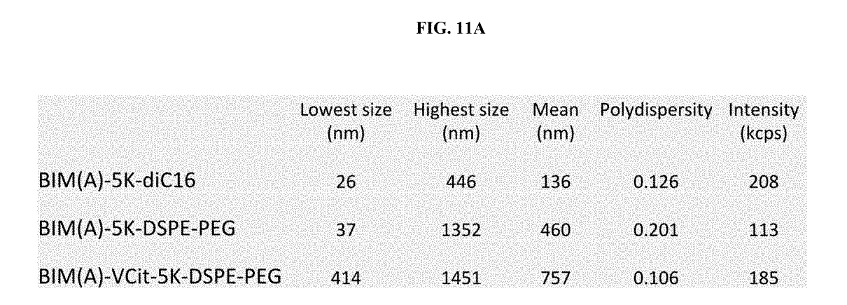

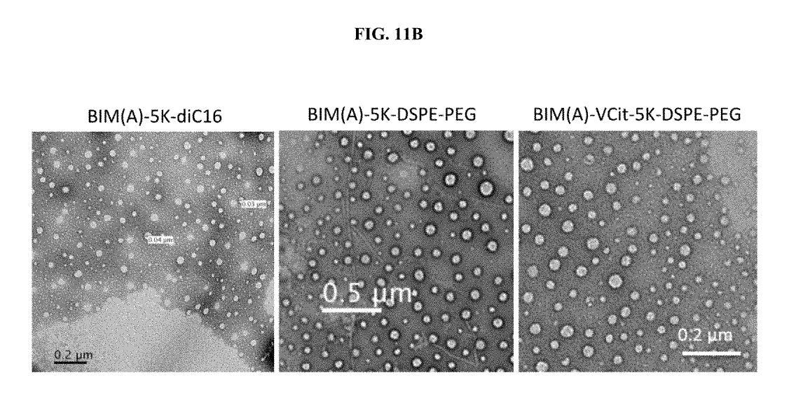

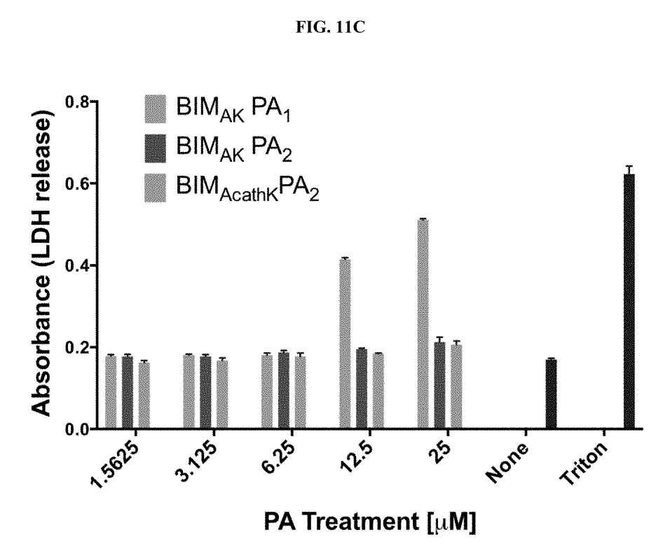

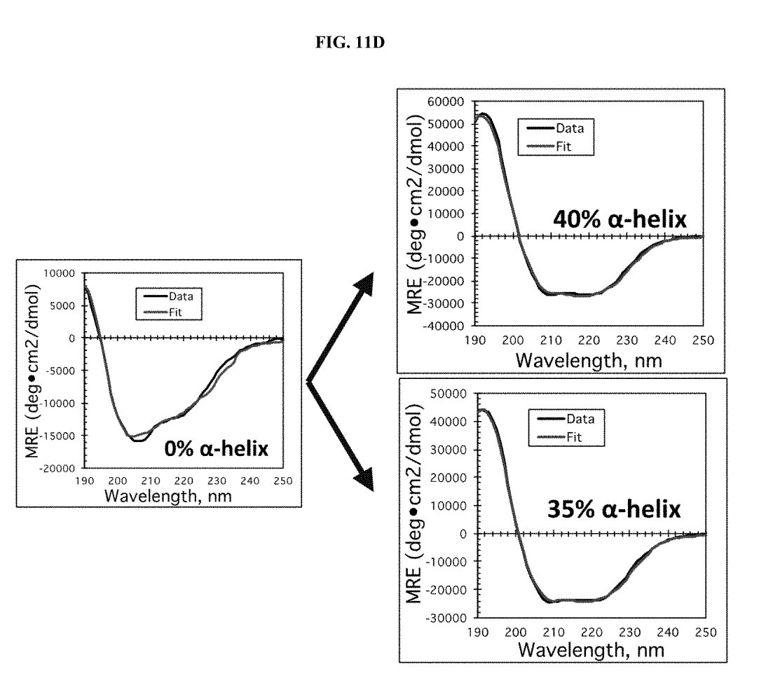

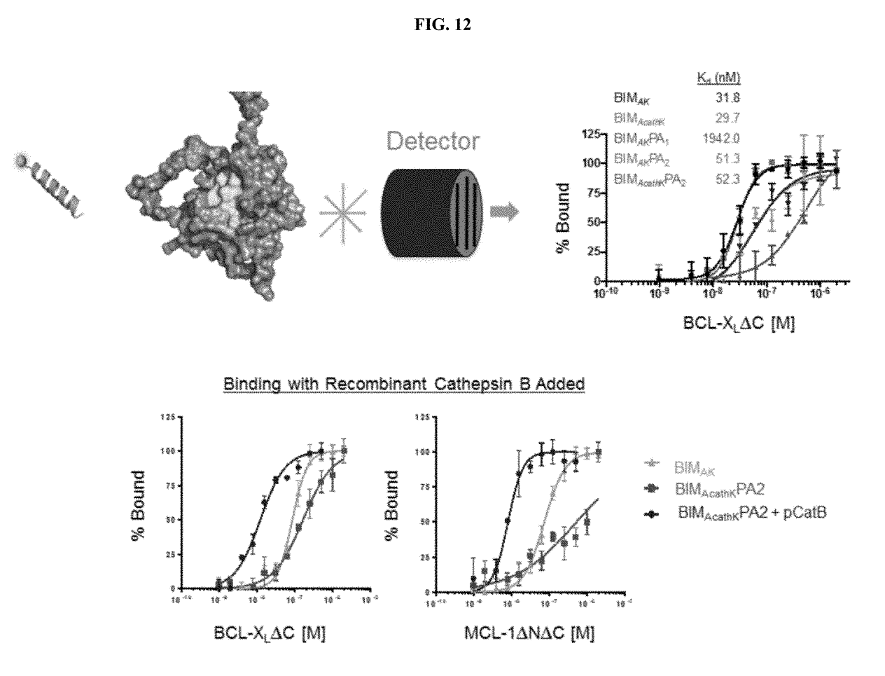

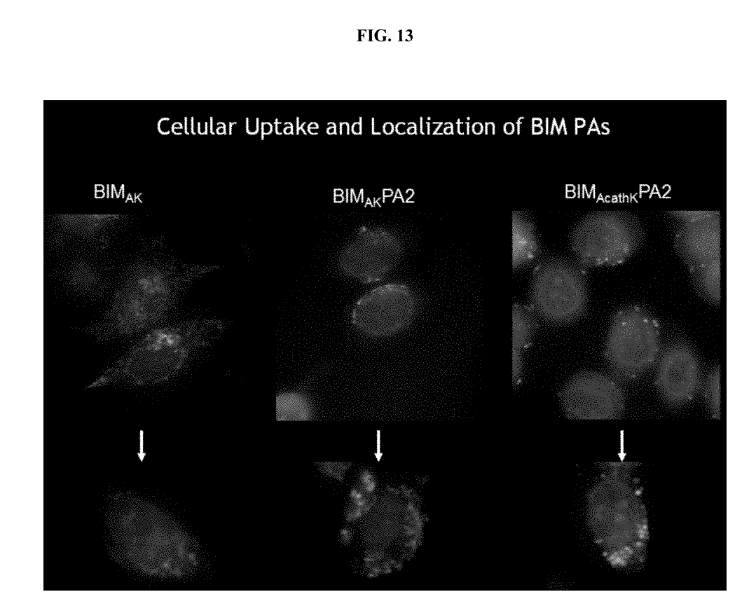

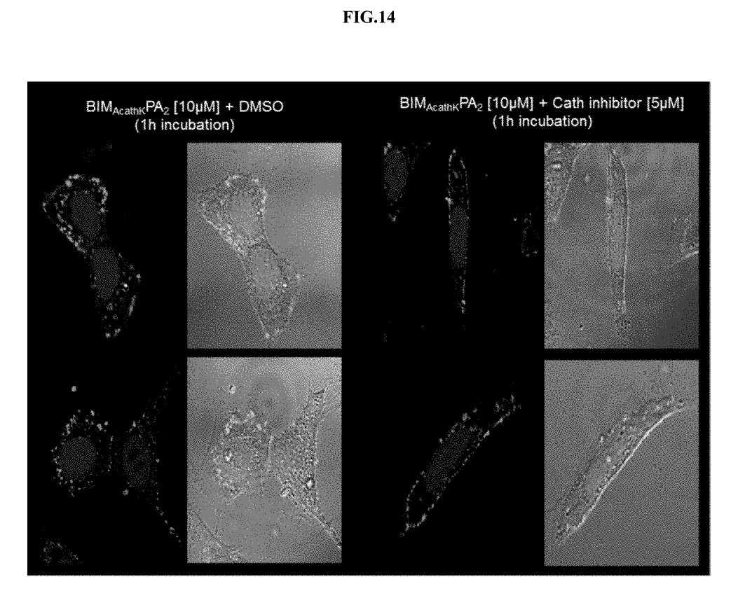

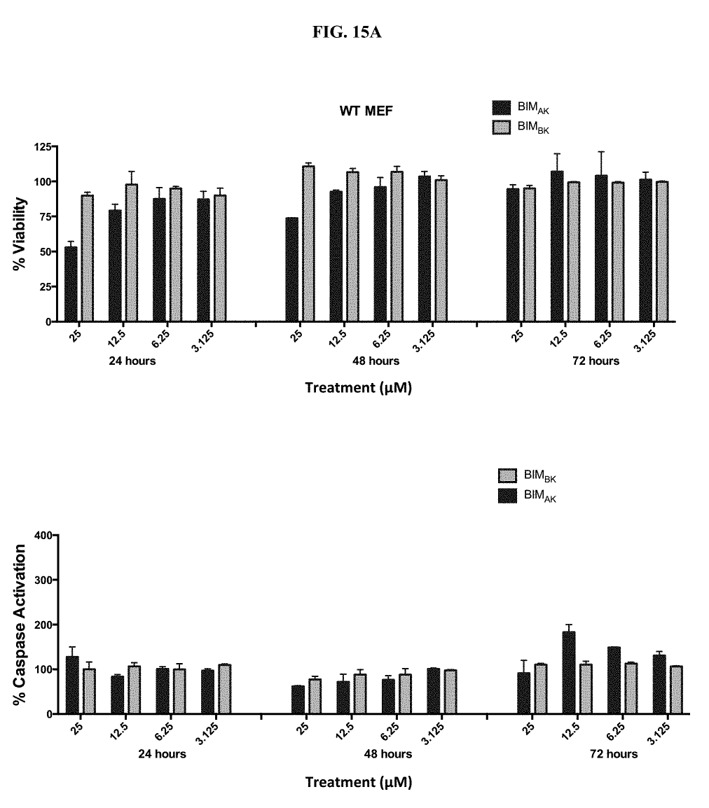

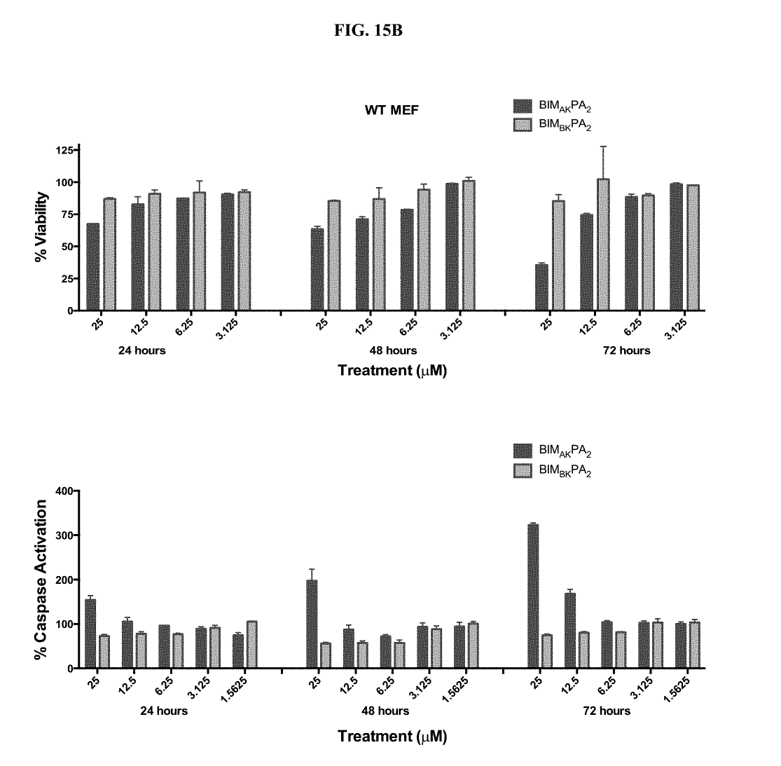

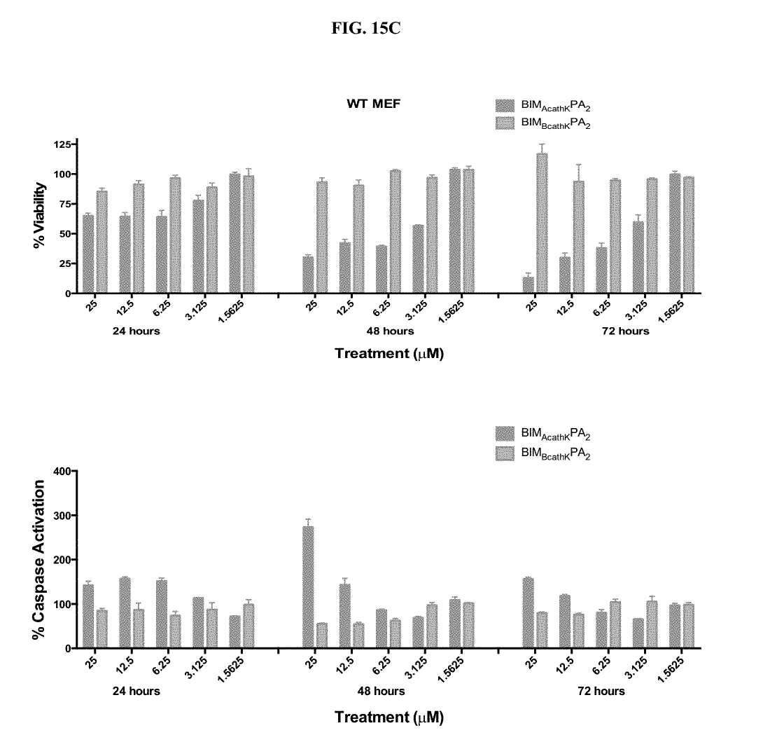

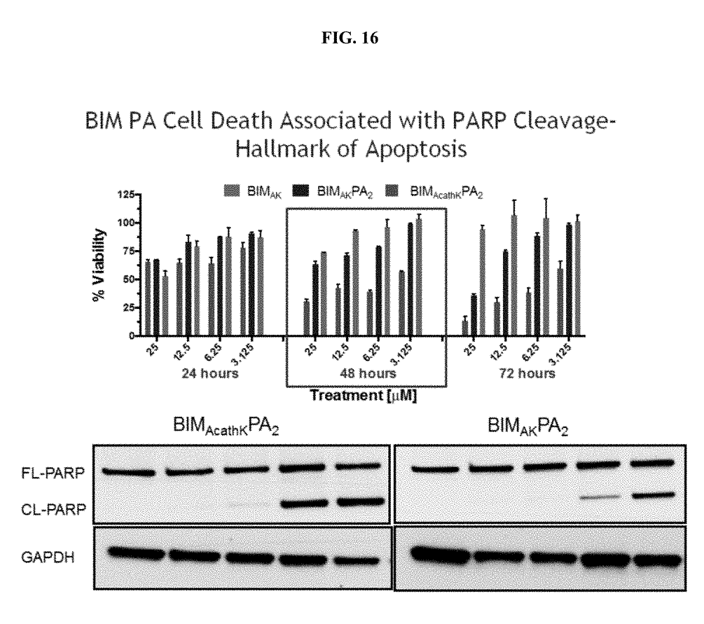

To determine if cathepsin cleavage and intracellular mapping would allow for complete dissociation of p53₍₁₄₋₂₉₎ from diC₁₆, catB-specific cleavage kinetics were measured in situ using recombinant human catB. The experimentally cleavable VC-PABA sequence and a control, non-cleavable triple glycine (GGG) linker, were conjugated to a 7-amino-4-methylcoumarin (AMC) dye, useful in studying protease activity and specificity (VC-PABA-AMC and GGG-AMC) (FIGS. 9A and 9B) (Ref. B45; incorporated by reference in its entirety). The electron group of the AMC fluorophore is localized, and thus remains quenched, when linked to the VC-PABA or GGG peptide substrate. When the covalent bond between the peptide and AMC is cleaved, this group de-localizes resulting in fluorescence detected at 440 nm (excitation: 348 nm) allowing for real-time measurement of enzyme/substrate kinetics. The addition of PABA improved catB-mediated cleavage of VC from AMC, likely through its well established spacer effect allowing catB ample access to the peptide substrate (FIG. 9C) (Ref. B46; incorporated by reference in its entirety). Fluorescence intensity of VC-PABA-AMC was rapidly increased compared to VC alone (FIG. 1C) (Refs. B34, B36; incorporated by reference in their entireties). Neither GGG-AMC or GG-PABA-AMC showed any change in fluorophore intensity indicating that PABA alone does not facilitate catB cleavage. GGG was used as a control sequence due to its similar peptide length with VC-PABA and its non-reactive side groups.

### Example 2

### CatB-Cleavable PAs with FRET Chromophores

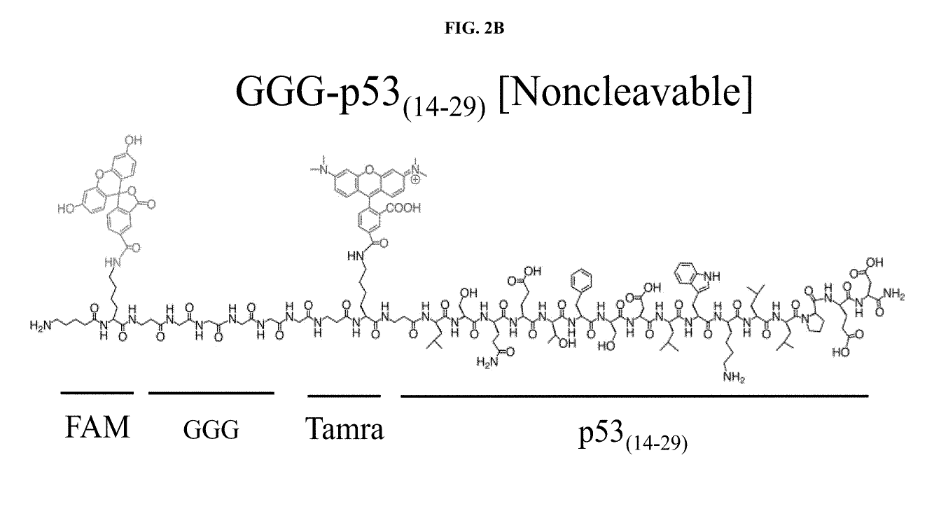

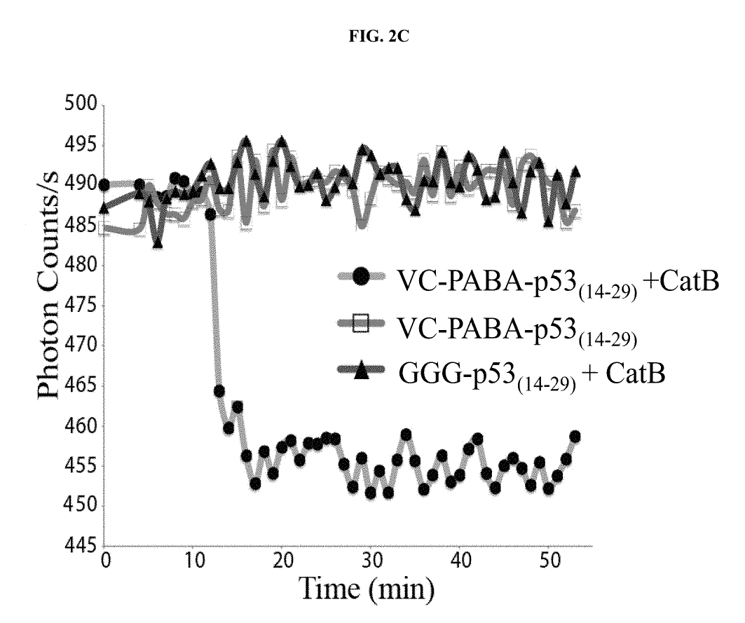

Experiments were conducted during development of embodiments herein to determine if catB cleavage fidelity and kinetics would transfer to intact PA monomers. To determine transit time and location of individual PA components, FAM (Fluorescein) and Tamra (Rhodamine) were placed on either side of the VC-PABA or GGG spacer. The fluorophores were located approximately 35.5 Å and 35.1 Å apart respectively with Tamra labeling the N-terminus of p53₍₁₄₋₂₉₎ and FAM labeling the N-terminus of either valine or glycine (FIG. 2A). The length between FAM and Tamra FRET fluorophores on the amino acid linkers between VC-PABA-p53₍₁₄₋₂₉₎ is 35.5 Å, and GGG-p53₍₁₄₋₂₉₎ is 35.1 Å. FAM (donor) excitation at 488 nm causes emission at 520 nm that in turn excites Tamra (acceptor) that emits a “FRET” wavelength of 620 nm. The efficiency of this energy transfer (FRET efficiency) is extremely sensitive to the small changes in distance within 10 nm of one another (ref. B47; incorporated by reference in its entirety). A change in intensity of the emitted light at 620 nm after excitation at 488 nm would thus reflect dissociation of p53₍₁₄₋₂₉₎ from the PA hydrophobic tail. (FIG. 1).

To ultimately monitor enzymatic cleavage of intact PA monomers FRET efficiency was first measured using wide field microscopy on p53₍₁₄₋₂₉₎ peptides with N-terminally located catB sequences with corresponding fluorochromes. To insure that the p53₍₁₄₋₂₉₎ peptide did not induce apoptosis in later cellular trafficking studies, the native conformer of p53₍₁₄₋₂₉₎ was used. Native p53₍₁₄₋₂₉₎ cannot enter cells and binds MDM2/4 less avidly than α-helical-reinforced peptides thus ensuring that the driving force for intracellular PA translocation is diC₁₆ and that native p53 is not activated thus allowing treated cells to live long enough for trafficking analysis (FIGS. 2A and 2B) (Refs. B15, B19, B33; incorporated by reference in their entireties). Peptides on resin were incubated on a platform with incoming light fixed at a diameter of 200 nm allowing the measurement of a localized bead area using 100× magnification. Using this method, the acceptor intensity (Tamra) diminished significantly following catB addition in the VC-PABA-p53₍₁₄₋₂₉₎ while there was no difference in FRET signal when catB was not added or when added to the non-cleavable control peptide (FIG. 2C). To ensure that catB did not significantly affect p53₍₁₄₋₂₉₎, media was collected at 3 and 24 hours following addition of catB and individual FAM and Tamra fluorescence measured. Increased FAM emission (N-terminal to the cleavage site) indicates successful FAM dissociation while Tamra emission indicates enzymolysis of internal p53₍₁₄₋₂₉₎ amino acids. FAM intensity in the media following catB addition to VC-PABA-p53₍₁₄₋₂₉₎ was significantly higher than that measured in the supernatant from GGG-p53₍₁₄₋₂₉₎ indicating efficient catB-directed cleavage from PA monomers. Tamra fluorescence in the media was minimal for both compounds indicating relative in vitro stability of the p53₍₁₄₋₂₉₎ peptide. It was next tested if this p53₍₁₄₋₂₉₎ catB resistance enables measurement of peptide accumulation inside cells.

### Example 3

### Intracellular Accumulation of PA Components

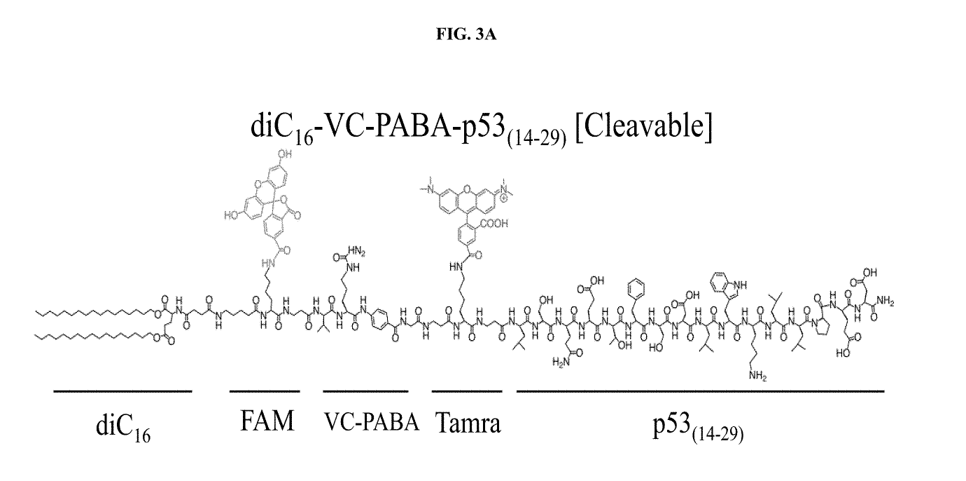

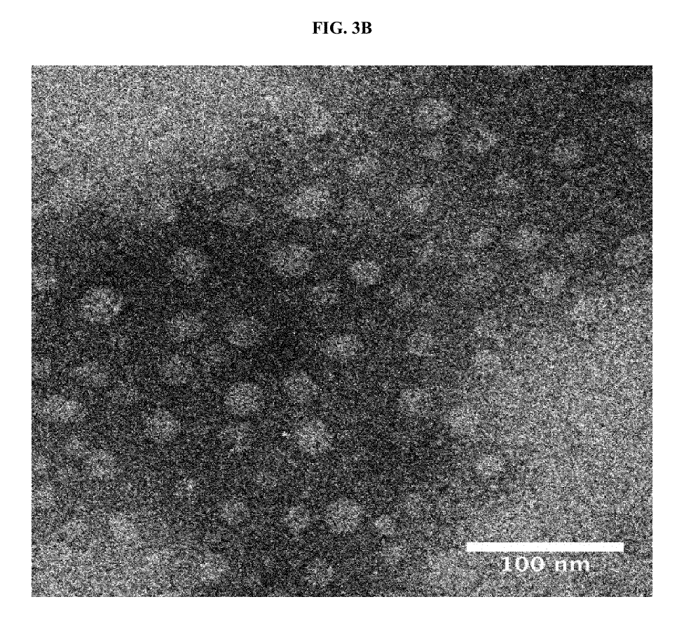

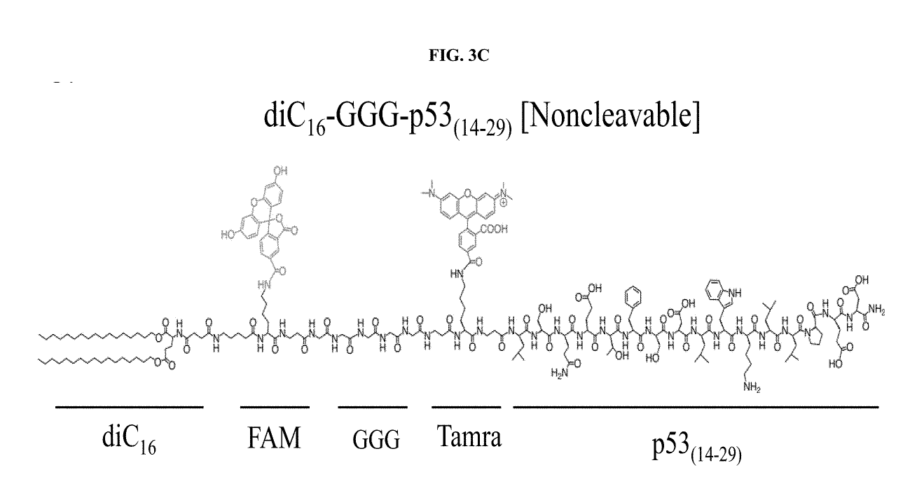

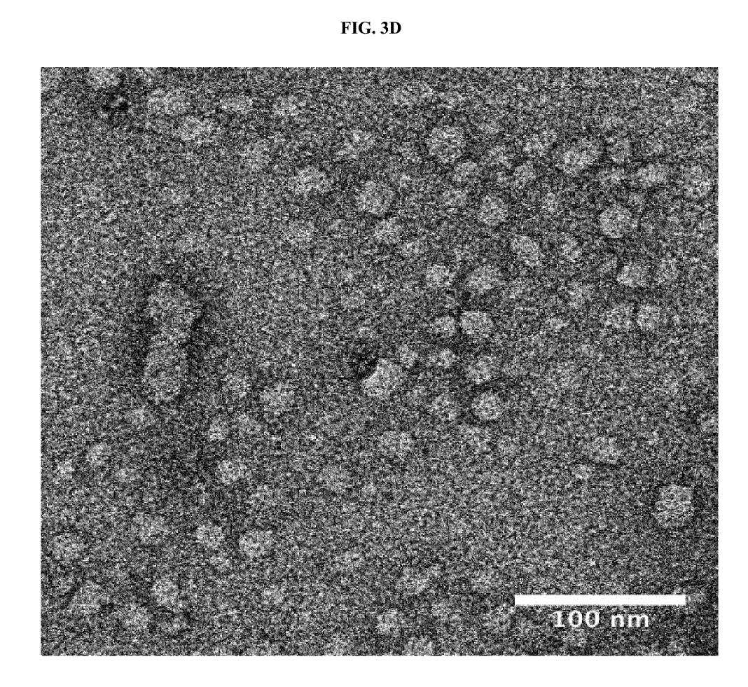

Building from the in vitro testing, p53₍₁₄₋₂₉₎ cleavable (diC₁₆-VC-PABA-p53₍₁₄₋₂₉₎) and non-cleavable (diC₁₆-GGG-p53₍₁₄₋₂₉₎) PAs were synthesized using the diC₁₆ hydrophobic tail (FIG. 3). Both PA micelles were of similar size and critical micellar concentrations (CMC) allowing for valid comparisons of treatment doses. The diC₁₆-p53₍₁₄₋₂₉₎ PAs formed spherical micelles between 20-40 nm (with occasional larger aggregates) as measured by transmission electron microscopy (TEM) and dynamic light scattering (DLS) (FIG. 3). Dynamic light scattering (DLS) of diC₁₆-VC-PABA-p53₍₁₄₋₂₉₎ and diC₁₆-GGG-p53₍₁₄₋₂₉₎ PAs indicate predominant scattering of micelles between 10-40 nm. A second scattering at 150 nm (diC₁₆-VC-PABA-p53₍₁₄₋₂₉₎) and 200 nm (diC₁₆-GGG-p53₍₁₄₋₂₉₎) is likely secondary to PA aggregation secondary to the FAM and Tamra hydrophobic dyes. Critical micelle concentration for PA micelles are similar. Although the DLS size distribution suggests that the PAs could exist in either micelle or rod-like transition, only round micelles were seen in TEM. This difference in secondary structure likely resulted from the additional amino acids and fluorochromes between diC₁₆ and p53₍₁₄₋₂₉₎ elongating the polar PA headgroup and driving round micelle formation through electrostatic repulsion (Refs. B26, B48-B50; incorporated by reference in their entireties). The catB cleavage analysis of these PA monomers was repeated and confirmed separation of diC₁₆ from p53₍₁₄₋₂₉₎ only after addition of recombinant catB using LCMS.

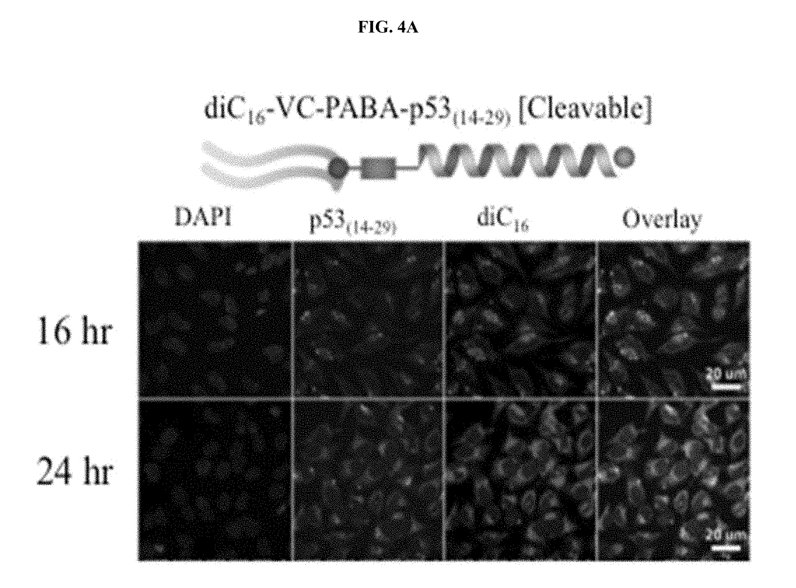

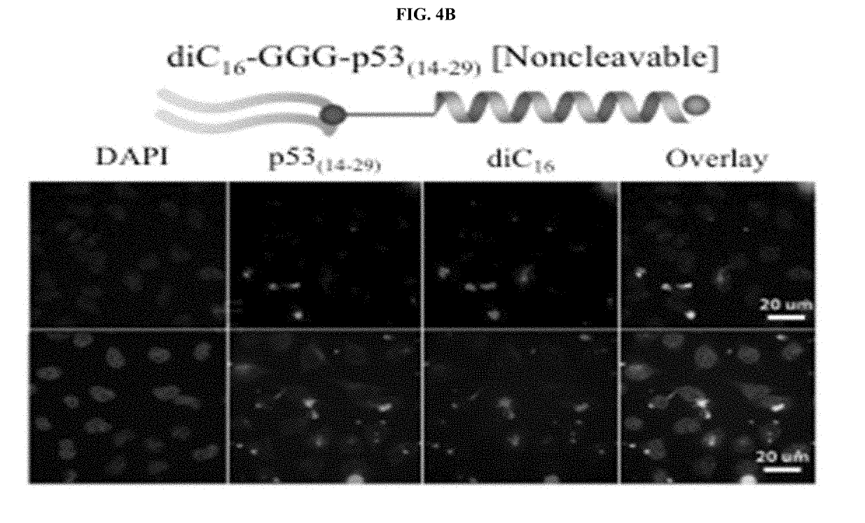

To determine long-term diC₁₆-VC-PABA-p53₍₁₄₋₂₉₎ and diC₁₆-GGG-p53₍₁₄₋₂₉₎ intracellular accumulation under continuous PA incubation, FAM and Tamra were moved out of FRET overlap range (FIG. 4). By moving the fluorochromes away from one another experiments were able to determine individual component accumulation without FRET interference. DLS found these PAs similar to the those detailed in FIG. 3 although slightly larger, between 50-100 nm, and with CMCs of 4.7 μM and 5.8 μM respectively. HeLa cells where incubated with 10 μM PA where p53₍₁₄₋₂₉₎ was C-terminally labeled with FAM and diC₁₆ C-terminally labeled with Tamra. Intracellular accumulation of diC₁₆-VC-PABA-p53₍₁₄₋₂₉₎ PA at 16 and 24 hours was far greater than non-cleavable control PAs (FIG. 4). Cells incubated with cleavable PAs accumulated diC₁₆ diffusely throughout the cells while discrete puncture of p53₍₁₄₋₂₉₎ overlapped considerably with diC₁₆ in cells incubated with diC₁₆-GGG-p53₍₁₄₋₂₉₎ (FIG. 4). However, due to the intense accumulation over time, it was impossible to determine if p53₍₁₄₋₂₉₎ had been cleaved from diC₁₆ in cells incubated with diC₁₆-VC-PABA-p53₍₁₄₋₂₉₎. Although initially internalized, non-cleavable PAs did not intracellularly accumulate over time (e.g., FIG. 4). This may have been because of recycling out of the cell or sequestration by FBS in the culture serum (Refs. B29, B51; incorporated by reference in their entireties). Because therapeutic peptide accumulation within target cells is necessary to obtain effective clinical responses, experiments were conducted during development of embodiments herein to determine if the p53₍₁₄₋₂₉₎ peptide was cleaved from PA monomers and at what time this occurred following internalization.

### Example 4

### PA Component Intracellular Cleavage and Trafficking

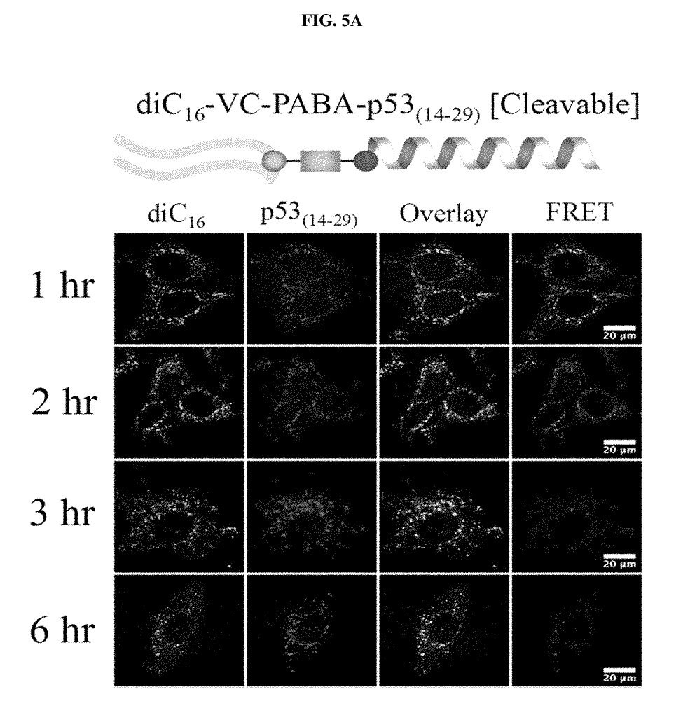

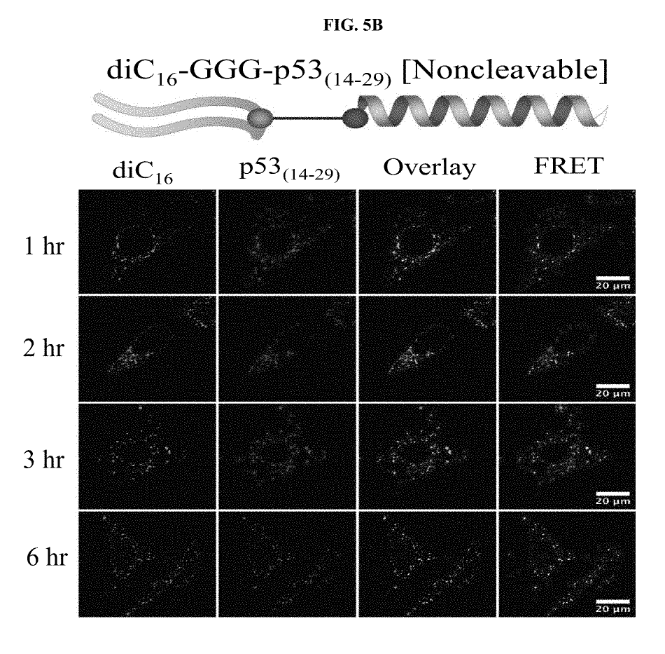

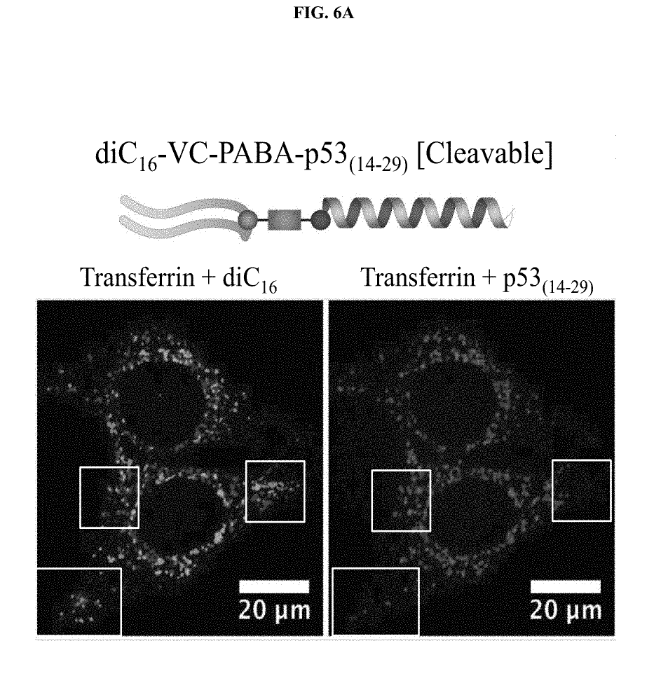

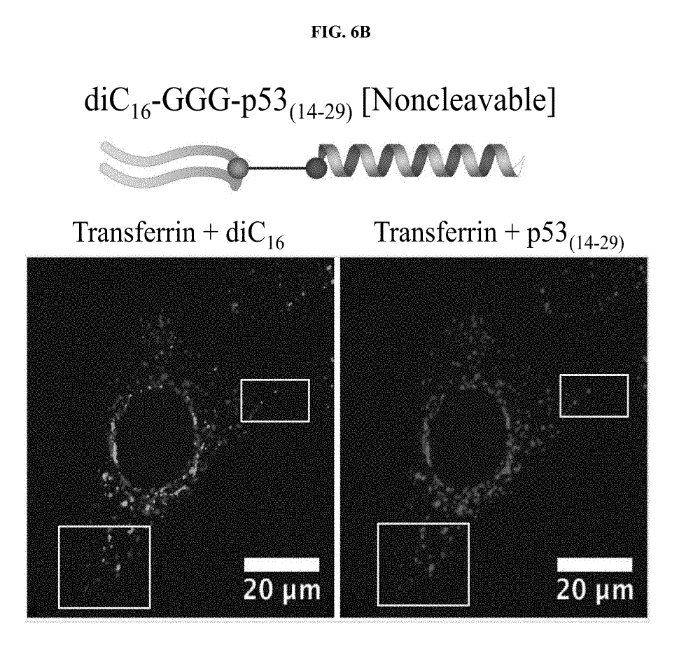

To better understand trafficking of p53₍₁₄₋₂₉₎ inside cells, FRET capable PA constructs were used (FIG. 3). HeLa cells were pulsed with 2.5 μM PA for 1 hour and washed rather than allow for continuous PA exposure that would complicate our visualization of catB-mediated cleavage and intracellular trafficking. Both PAs were equivalently internalized within one hour of incubation (FIG. 5). Each was taken in through endocytosis with substantial compartmental co-localization with transferrin-positive intracellular vesicles, reflective of early and late endosomal trafficking (FIG. 6). Despite most intact PAs being associated with transferrin-positive early endosomes, there was also evidence of dissociation and movement of diC₁₆ out of these endosomes and into other areas of the cell as early as 1 hour following treatment with diC₁₆-VC-PABA-p53₍₁₄₋₂₉₎ (FIG. 6). Transferrin labels early endosomes that ultimately transition to sorting endosomes or endocytic recycling compartments where transferrin is released from the transferrin receptor at low pH (Refs. B52-B54; incorporated by reference in their entireties). Because this process can take as little as 10 minutes, it is unclear if these diC₁₆ tail fragments were located within late endosomes or being recycled back to the cell surface (Ref. B54; incorporated by reference in its entirety). Regardless, unlike diC₁₆-VC-PABA-p53₍₁₄₋₂₉₎, control diC₁₆-GGG-p53₍₁₄₋₂₉₎ PAs were universally found in vesicles as one unit (FIGS. 5 and 6). Cleavage of p53₍₁₄₋₂₉₎ from diC₁₆ and loss of FRET signal in cells treated diC₁₆-VC-PABA-p53₍₁₄₋₂₉₎ occurred almost completely by 3 hours following incubation whereas FRET signal was retained in cells treated with diC₁₆-GGG-p53₍₁₄₋₂₉₎ PAs (FIG. 5). p53₍₁₄₋₂₉₎ peptide appeared to accumulate in discrete locations within the cell by 6 hours in contrast to control PAs. Additionally, diC₁₆-GGG-p53₍₁₄₋₂₉₎ PA treated cells lost overall intensity over time (FIG. 4). The diffuse spreading of diC₁₆ throughout the cell after treatment with diC₁₆-VC-PABA-p53₍₁₄₋₂₉₎ indicates that these compartments are destined for exocytosis and/or membrane recycling (FIG. 5A) (Refs. B20, B29, B51, B54; incorporated by reference in their entireties). The apparent decrease in tail/peptide signal intensity in cells treated with diC₁₆-GGG-p53₍₁₄₋₂₉₎ PAs indicates that intact PA monomers are ejected from the cell over time (FIG. 5B).

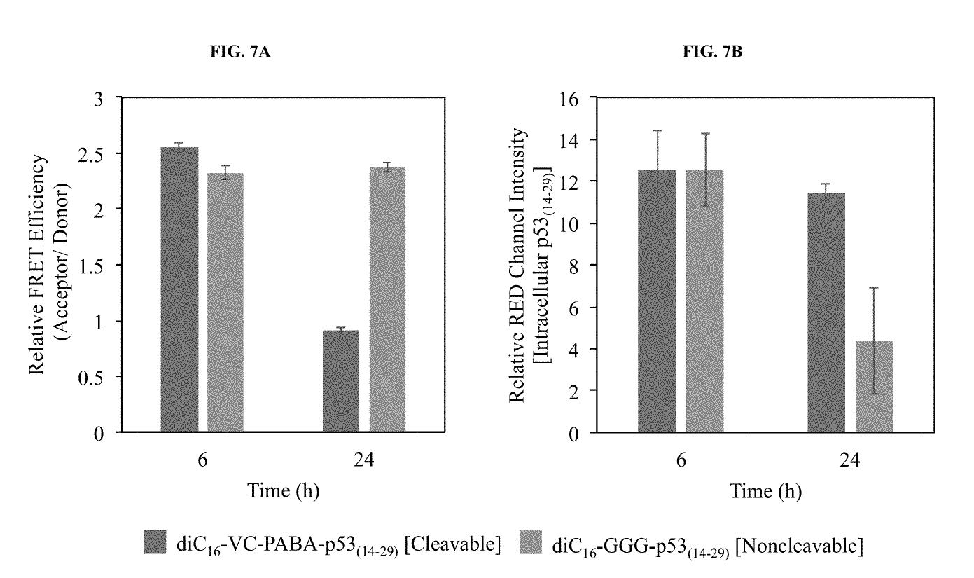

To confirm that loss of FRET signal was due to cleavage of p53₍₁₄₋₂₉₎ from diC₁₆ and not loss of FRET efficiency (e.g., due to loss of fluorescent intensity, photobleaching, etc.), HeLa cells were treated as above but with an increased PA concentration of 10 μM. Extracellular PA was washed away after 1 hour and cells were allowed to incubate for 6 and 24 hours followed by superresolution laser scanning confocal microscopy. Raw images were analyzed for FRET signaling at each time point comparing diC₁₆-VC-PABA-p53₍₁₄₋₂₉₎ to diC₁₆-GGG-p53₍₁₄₋₂₉₎ and non-treated cells (FIG. 7). While FRET signaling decreased from 6 hours to 24 hours in cells treated with diC₁₆-VC-PABA-p53₍₁₄₋₂₉₎ (FIG. 5) there was no change in FRET efficiency of diC₁₆-GGG-p53₍₁₄₋₂₉₎-treated cells. Therefore cleavage of p53₍₁₄₋₂₉₎ from diC₁₆ and movement of p53₍₁₄₋₂₉₎ to spatially distinct areas of the cell occurred only in relation to diC₁₆-VC-PABA-p53₍₁₄₋₂₉₎ and was not due to loss of the ability of intact PAs to provide a quantifiable FRET signal over time (FIG. 7A).

To quantify the amount of p53₍₁₄₋₂₉₎ peptide in individual cells following incubation for 24 hours, Tamra intensity alone was measured at 520 nm excitation and fluorescence at 580-660 nm. Using this method, the relative amount of intracellular p53₍₁₄₋₂₉₎ peptide was similar between diC₁₆-VC-PABA-p53₍₁₄₋₂₉₎ and diC₁₆-GGG-p53₍₁₄₋₂₉₎-treated cells at 6 hours (FIG. 7B). However, by 24 hours p53₍₁₄₋₂₉₎ peptide levels dropped significantly in cells treated with diC₁₆-GGG-p53₍₁₄₋₂₉₎ confirming the results under continuous treatment conditions (FIG. 4). A source of this decrease over time is that diC₁₆ leads to endosomal membrane tethering and facilitates recycling of the intact diC₁₆-GGG-p53₍₁₄₋₂₉₎PA monomers out of the cells.

### Example 5

### Extracellular Trafficking of Intact PA Monomers and Individual PA Components

Given the rapid intracellular trafficking of PAs, experiments were conducted during development of embodiments herein to determine if overall loss of diC₁₆-GGG-p53₍₁₄₋₂₉₎ monomers or diC₁₆ from diC₁₆-VC-PABA-p53₍₁₄₋₂₉₎-treated cells was due to membrane recycling and extrusion via extracellular vesicles. The hydrophobic tails of PAs are thought to promote lipid membrane anchoring of PA monomers and subsequent membrane tethering (Refs. B20, B27, B51, B55; incorporated by reference in their entireties). Membrane invaginations during endocytosis therefore contain these and other extracellular lipids that are transported through the endo-lysomal pathway and either metabolized through autophagocytosis or refluxed out of the cell within extracellular vesicles (Refs. B56-B57; incorporated by reference in their entireties).

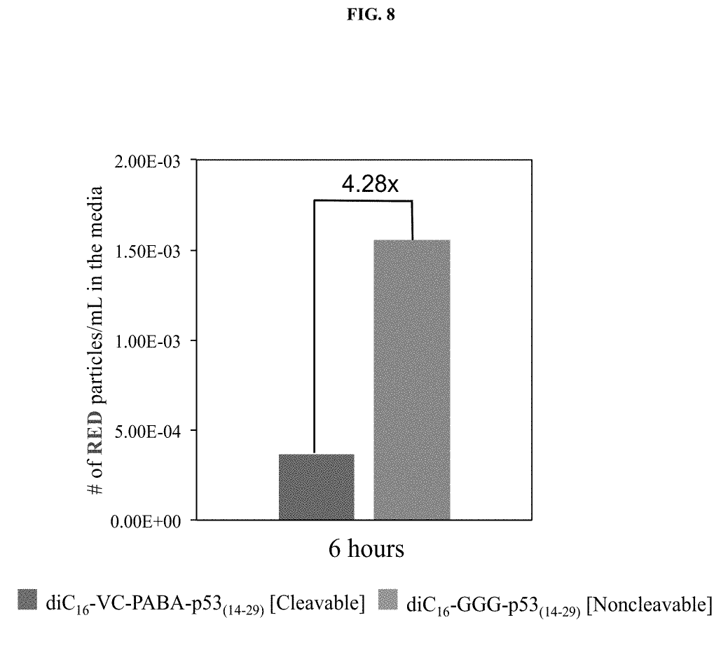

To measure extracellular vesicles, HeLa cells were incubated for 1 hour followed by washing and replacement with PA-free media. The cells were then allowed to incubate and media was collected at 6 hours following incubation. Vesicles within the media were analyzed using a Nanosight N300 with fluorescence filters and nanoparticle tracking analysis (NTA) software was used to determine the number of total and red particles per frame over a threshold of a constant intensity (FIG. 8). The number of red extracellular particles were less in media from cells incubated with diC₁₆-VC-PABA-p53₍₁₄₋₂₉₎ compared to cells treated with diC₁₆-GGG-p53₍₁₄₋₂₉₎ PAs supporting efflux of intact diC₁₆-GGG-p53₍₁₄₋₂₉₎ and either intracellular accumulation (FIGS. 4-6) or metabolism of p53₍₁₄₋₂₉₎ peptides. The number of peptides within each vesicle could not be determined using this technique. Coincident measurement of diC₁₆-laded vesicles could also not be performed accurately due to limitations in the Nanosight laser/detector thresholds. Despite these limitations, these results indicate that the hydrophobic diC₁₆ tails drive excretion/recycling of intact PA monomers in the systems described herein.

### REFERENCES

The following references, some of which are cited above by number, are herein incorporated by reference in their entireties:
A1. Li, Y. Cathepsin B-cleavable doxorubicin prodrugs for targeted cancer therapy (Review). Int J Oncol (2012). doi:10.3892/ijo.2012.1754
A2. Calderón, M., Graeser, R., Kratz, F. & Haag, R. Development of enzymatically cleavable prodrugs derived from dendritic polyglycerol. Bioorganic & Medicinal Chemistry Letters 19, 3725-3728 (2009).
A3. Thanou, M. & Duncan, R. Polymer-protein and polymer-drug conjugates in cancer therapy. Curr Opin Investig Drugs 4, 701-709 (2003).
A4. Duncan, R., Cable, H. C., Lloyd, J. B., Rejmanová, P. & Kope\check{c}ek, J. Degradation of side-chains ofN-(2-hydroxypropyl)methacrylamide copolymers by lysosomal thiol-proteinases. Biosci Rep 2, 1041-1046 (1982).
A5. Dubowchik, G. M. et al. Cathepsin B-Labile Dipeptide Linkers for Lysosomal Release of Doxorubicin from Internalizing Immunoconjugates: Model Studies of Enzymatic Drug Release and Antigen-Specific In Vitro Anticancer Activity. Bioconjugate Chem. 13, 855-869 (2002).
A6. Ková\check{r}, M., Strohalm, J., Etrych, T., Ulbrich, K. & \check{R}ihová, B. Star Structure of Antibody-Targeted HPMA Copolymer-Bound Doxorubicin: A Novel Type of Polymeric Conjugate for Targeted Drug Delivery with Potent Antitumor Effect. Bioconjugate Chem. 13, 206-215 (2002).
A7. Chakravarty, P. K. & Katzenellenbogen, J. A. A novel connector linkage applicable in prodrug design. Journal of Medicinal Chemistry (1981).
A8. Chowdhury, M. A. et al. Prodrug-Inspired Probes Selective to Cathepsin B over Other Cysteine Cathepsins. Journal of Medicinal Chemistry 57, 6092 (2014).
A9. Dorywalska, M. et al. Effect of Attachment Site on Stability of Cleavable Antibody Drug Conjugates. Bioconjugate . . . 26, 650-659 (2015).
A10. Lyon, R. P. et al. Reducing hydrophobicity of homogeneous antibody-drug conjugates improves pharmacokinetics and therapeutic index. Nature Biotechnology 33, 733-735 (2015).
A11. Anticancer drugs conjugated to antibody via an enzyme cleavable linker; WO Application: WO2007105027A8
A12. Lysosomal enzyme-cleavable antitumor drug conjugates. US Grant: U.S. Pat. No. 6,214,345B1
B1. London, N.; Raveh, B.; Schueler-Furman, O., Druggable protein-protein interactions—from hot spots to hot segments. Curr Opin Chem Biol 2013, 17 (6), 952-9.
B2. Rolland, T.; Tasan, M.; Charloteaux, B.; Pevzner, S. J.; Zhong, Q.; Sahni, N.; Yi, S.; Lemmens, I.; Fontanillo, C.; Mosca, R., et al., A proteome-scale map of the human interactome network. Cell 2014, 159 (5), 1212-26.
B3. Moll, U. M.; Wolff, S.; Speidel, D.; Deppert, W., Transcription-independent pro-apoptotic functions of p53. Curr Opin Cell Biol 2005, 17 (6), 631-6.
B4. Bieging, K. T.; Mello, S. S.; Attardi, L. D., Unravelling mechanisms of p53-mediated tumour suppression. Nat Rev Cancer 2014, 14 (5), 359-70.
B5. Brown, C. J.; Lain, S.; Verma, C. S.; Fersht, A. R.; Lane, D. P., Awakening guardian angels: drugging the p53 pathway. Nat Rev Cancer 2009, 9 (12), 862-73.
B6. Burgess, A.; Chia, K. M.; Haupt, S.; Thomas, D.; Haupt, Y.; Lim, E., Clinical Overview of MDM2/X-Targeted Therapies. Front Oncol 2016, 6, 7.
B7. Itahana, K.; Mao, H.; Jin, A.; Itahana, Y.; Clegg, H. V.; Lindstrom, M. S.; Bhat, K. P.; Godfrey, V. L.; Evan, G. I.; Zhang, Y., Targeted inactivation of Mdm2 RING finger E3 ubiquitin ligase activity in the mouse reveals mechanistic insights into p53 regulation. Cancer Cell 2007, 12 (4), 355-66.
B8. Parant, J.; Chavez-Reyes, A.; Little, N. A.; Yan, W.; Reinke, V.; Jochemsen, A. G.; Lozano, G., Rescue of embryonic lethality in Mdm4-null mice by loss of Trp53 suggests a nonoverlapping pathway with MDM2 to regulate p53. Nat Genet 2001, 29 (1), 92-5.
B9. Bond, G. L.; Hu, W.; Levine, A., A single nucleotide polymorphism in the MDM2 gene: from a molecular and cellular explanation to clinical effect. Cancer Res 2005, 65 (13), 5481-4.
B10. Ohtsubo, C.; Shiokawa, D.; Kodama, M.; Gaiddon, C.; Nakagama, H.; Jochemsen, A. G.; Taya, Y.; Okamoto, K., Cytoplasmic tethering is involved in synergistic inhibition of p53 by Mdmx and Mdm2. Cancer Sci 2009, 100 (7), 1291-9.
B11. Vousden, K. H.; Lane, D. P., p53 in health and disease. Nat Rev Mol Cell Biol 2007, 8 (4), 275-83.
B12. Monti, S.; Chapuy, B.; Takeyama, K.; Rodig, S. J.; Hao, Y.; Yeda, K. T.; Inguilizian, H.; Mermel, C.; Currie, T.; Dogan, A., et al., Integrative analysis reveals an outcome-associated and targetable pattern of p53 and cell cycle deregulation in diffuse large B cell lymphoma. Cancer Cell 2012, 22 (3), 359-72.
B13. Morin, R. D.; Mendez-Lago, M.; Mungall, A. J.; Goya, R.; Mungall, K. L.; Corbett, R. D.; Johnson, N. A.; Severson, T. M.; Chiu, R.; Field, M., et al., Frequent mutation of histone-modifying genes in non-Hodgkin lymphoma. Nature 2011, 476 (7360), 298-303.
B14. Acar, H.; Srivastava, S.; Chung, E. J.; Schnorenberg, M. R.; Barrett, J. C.; LaBelle, J. L.; Tirrell, M., Self-assembling peptide-based building blocks in medical applications. Adv Drug Deliv Rev 2016, 110-111, 65-79.
B15. Bernal, F.; Wade, M.; Godes, M.; Davis, T. N.; Whitehead, D. G.; Kung, A. L.; Wahl, G. M.; Walensky, L. D., A stapled p53 helix overcomes HDMX-mediated suppression of p53. Cancer Cell 2010, 18 (5), 411-22.
B16. Shangary, S.; Ding, K.; Qiu, S.; Nikolovska-Coleska, Z.; Bauer, J. A.; Liu, M.; Wang, G.; Lu, Y.; McEachern, D.; Bernard, D., et al., Reactivation of p53 by a specific MDM2 antagonist (MI-43) leads to p21-mediated cell cycle arrest and selective cell death in colon cancer. Mol Cancer Ther 2008, 7 (6), 1533-42.
B17. Shangary, S.; Qin, D.; McEachern, D.; Liu, M.; Miller, R. S.; Qiu, S.; Nikolovska-Coleska, Z.; Ding, K.; Wang, G.; Chen, J., et al., Temporal activation of p53 by a specific MDM2 inhibitor is selectively toxic to tumors and leads to complete tumor growth inhibition. Proc Natl Acad Sci USA 2008, 105 (10), 3933-8.
B18. Vassilev, L. T.; Vu, B. T.; Graves, B.; Carvajal, D.; Podlaski, F.; Filipovic, Z.; Kong, N.; Kammlott, U.; Lukacs, C.; Klein, C., et al., In vivo activation of the p53 pathway by small-molecule antagonists of MDM2. Science 2004, 303 (5659), 844-8.
B19. Bernal, F.; Tyler, A. F.; Korsmeyer, S. J.; Walensky, L. D.; Verdine, G. L., Reactivation of the p53 tumor suppressor pathway by a stapled p53 peptide. J Am Chem Soc 2007, 129 (9), 2456-7.
B20. Missirlis, D.; Krogstad, D. V.; Tirrell, M., Internalization of p53(14-29) peptide amphiphiles and subsequent endosomal disruption results in SJSA-1 cell death. Mol Pharm 2010, 7 (6), 2173-84.
B21. Chang, Y. S.; Graves, B.; Guerlavais, V.; Tovar, C.; Packman, K.; To, K. H.; Olson, K. A.; Kesavan, K.; Gangurde, P.; Mukherjee, A., et al., Stapled alpha-helical peptide drug development: a potent dual inhibitor of MDM2 and MDMX for p53-dependent cancer therapy. Proc Natl Acad Sci USA 2013, 110 (36), E3445-54.
B22. Wells, J. A.; McClendon, C. L., Reaching for high-hanging fruit in drug discovery at protein-protein interfaces. Nature 2007, 450 (7172), 1001-9.
B23. Azzarito, V.; Long, K.; Murphy, N. S.; Wilson, A. J., Inhibition of alpha-helix-mediated protein-protein interactions using designed molecules. Nat Chem 2013, 5 (3), 161-73.
B24. Clackson, T.; Wells, J. A., A hot spot of binding energy in a hormone-receptor interface. Science 1995, 267 (5196), 383-6.
B25. Lim, Y. B.; Lee, E.; Lee, M., Cell-penetrating-peptide-coated nanoribbons for intracellular nanocarriers. Angew Chem Int Edit 2007, 46 (19), 3475-3478.
B26. Trent, A.; Marullo, R.; Lin, B.; Black, M.; Tirrell, M., Structural properties of soluble peptide amphiphile micelles. Soft Matter 2011, 7 (20), 9572-9582.
B27. Missirlis, D.; Chworos, A.; Fu, C. J.; Khant, H. A.; Krogstad, D. V.; Tirrell, M., Effect of the peptide secondary structure on the peptide amphiphile supramolecular structure and interactions. Langmuir 2011, 27 (10), 6163-70.
B28. Blanco, E.; Shen, H.; Ferrari, M., Principles of nanoparticle design for overcoming biological barriers to drug delivery. Nat Biotechnol 2015, 33 (9), 941-51.
B29. Missirlis, D.; Teesalu, T.; Black, M.; Tirrell, M., The non-peptidic part determines the internalization mechanism and intracellular trafficking of peptide amphiphiles. PLoS One 2013, 8 (1), e54611.
B30. Duncan, R.; Richardson, S. C., Endocytosis and intracellular trafficking as gateways for nanomedicine delivery: opportunities and challenges. Mol Pharm 2012, 9 (9), 2380-402.
B31. Yu, B.; Tai, H. C.; Xue, W.; Lee, L. J.; Lee, R. J., Receptor-targeted nanocarriers for therapeutic delivery to cancer. Mol Membr Biol 2010, 27 (7), 286-98.
B32. Thanou, M.; Duncan, R., Polymer-protein and polymer-drug conjugates in cancer therapy. Curr Opin Investig Drugs 2003, 4 (6), 701-9.
B33. Li, Y. C.; Rodewald, L. W.; Hoppmann, C.; Wong, E. T.; Lebreton, S.; Safar, P.; Patek, M.; Wang, L.; Wertman, K. F.; Wahl, G. M., A versatile platform to analyze low-affinity and transient protein-protein interactions in living cells in real time. Cell Rep 2014, 9 (5), 1946-58.
B34. Dubowchik, G. M.; Firestone, R. A.; Padilla, L.; Willner, D.; Hofstead, S. J.; Mosure, K.; Knipe, J. O.; Lasch, S. J.; Trail, P. A., Cathepsin B-labile dipeptide linkers for lysosomal release of doxorubicin from internalizing immunoconjugates: model studies of enzymatic drug release and antigen-specific in vitro anticancer activity. Bioconjug Chem 2002, 13 (4), 855-69.
B35. Dorywalska, M.; Strop, P.; Melton-Witt, J. A.; Hasa-Moreno, A.; Farias, S. E.; Galindo Casas, M.; Delaria, K.; Lui, V.; Poulsen, K.; Loo, C., et al., Effect of attachment site on stability of cleavable antibody drug conjugates. Bioconjug Chem 2015, 26 (4), 650-9.
B36. Dubowchik, G. M.; Firestone, R. A., Cathepsin B-sensitive dipeptide prodrugs. 1. A model study of structural requirements for efficient release of doxorubicin. Bioorganic & Medicinal Chemistry Letters 1998, 8 (23), 3341-3346.
B37. Lyon, R. P.; Bovee, T. D.; Doronina, S. O.; Burke, P. J.; Hunter, J. H.; Neff-LaFord, H. D.; Jonas, M.; Anderson, M. E.; Setter, J. R.; Senter, P. D., Reducing hydrophobicity of homogeneous antibody-drug conjugates improves pharmacokinetics and therapeutic index. Nat Biotechnol 2015, 33 (7), 733-5.
B38. Carter, P. J.; Senter, P. D., Antibody-drug conjugates for cancer therapy. Cancer J 2008, 14 (3), 154-69.
B39. Staben, L. R.; Koenig, S. G.; Lehar, S. M.; Vandlen, R.; Zhang, D.; Chuh, J.; Yu, S. F.; Ng, C.; Guo, J.; Liu, Y., et al., Targeted drug delivery through the traceless release of tertiary and heteroaryl amines from antibody-drug conjugates. Nat Chem 2016, 8 (12), 1112-1119.
B40. Bouchard, H.; Viskov, C.; Garcia-Echeverria, C., Antibody-drug conjugates-a new wave of cancer drugs. Bioorg Med Chem Lett 2014, 24 (23), 5357-63.
B41. Farias, S. E.; Strop, P.; Delaria, K.; Casas, M. G.; Dorywalska, M.; Shelton, D. L.; Pons, J.; Rajpal, A., Mass Spectrometric Characterization of Transglutaminase Based Site-Specific Antibody-Drug Conjugates. Bioconjugate Chem 2014, 25 (2), 240-250.
B42. Maniganda, S.; Sankar, V.; Nair, J. B.; Raghu, K. G.; Maiti, K. K., A lysosome-targeted drug delivery system based on sorbitol backbone towards efficient cancer therapy. Org Biomol Chem 2014, 12 (34), 6564-6569.
B43. Wang, Y.; Cheetham, A. G.; Angacian, G.; Su, H.; Xie, L.; Cui, H., Peptide-drug conjugates as effective prodrug strategies for targeted delivery. Adv Drug Deliv Rev 2016, 110-111, 112-126.
B44. Burkhart, D. J.; Barthel, B. L.; Post, G. C.; Kalet, B. T.; Nafie, J. W.; Shoemaker, R. K.; Koch, T. H., Design, synthesis, and preliminary evaluation of doxazolidine carbamates as prodrugs activated by carboxylesterases. J Med Chem 2006, 49 (24), 7002-12.
B45. Maly, D. J.; Huang, L.; Ellman, J. A., Combinatorial strategies for targeting protein families: Application to the proteases. Chembiochem 2002, 3 (1), 17-37.
B46. Turk, V.; Stoka, V.; Vasiljeva, O.; Renko, M.; Sun, T.; Turk, B.; Turk, D., Cysteine cathepsins: from structure, function and regulation to new frontiers. Biochim Biophys Acta 2012, 1824 (1), 68-88.
B47. Roy, R.; Hohng, S.; Ha, T., A practical guide to single-molecule FRET. Nat Methods 2008, 5 (6), 507-516.
B48. Law, B.; Weissleder, R.; Tung, C. H., Mechanism-based fluorescent reporter for protein kinase A detection. Chembiochem 2005, 6 (8), 1361-1367.
B49. Ochocki, J. D.; Mullen, D. G.; Wattenberg, E. V.; Distefano, M. D., Evaluation of a cell penetrating prenylated peptide lacking an intrinsic fluorophore via in situ click reaction. Bioorganic & Medicinal Chemistry Letters 2011, 21 (17), 4998-5001.
B50. Salim, M.; Minamikawa, H.; Sugimura, A.; Hashim, R., Amphiphilic designer nanocarriers for controlled release: from drug delivery to diagnostics. Medchemcomm 2014, 5 (11), 1602-1618.
B51. Missirlis, D.; Khant, H.; Tirrell, M., Mechanisms of Peptide Amphiphile Internalization by SJSA-1 Cells in Vitro. Biochemistry 2009, 48 (15), 3304-3314.
B52. Hopkins, C. R., Intracellular Routing of Transferrin and Transferrin Receptors in Epidermoid Carcinoma A431-Cells. Cell 1983, 35 (1), 321-330.
B53. Hopkins, C. R.; Trowbridge, I. S., Internalization and Processing of Transferrin and the Transferrin Receptor in Human Carcinoma A431-Cells. J Cell Biol 1983, 97 (2), 508-521
B54. Maxfield, F. R.; McGraw, T. E., Endocytic recycling. Nat Rev Mol Cell Biol 2004, 5 (2), 121-32.
B55. Zacharias, D. A.; Violin, J. D.; Newton, A. C.; Tsien, R. Y., Partitioning of lipid-modified monomeric GFPs into membrane microdomains of live cells. Science 2002, 296 (5569), 913-6.
B56. Jaishy, B.; Abel, E. D., Lipids, lysosomes, and autophagy. J Lipid Res 2016, 57 (9), 1619-35.
B57. van Meer, G.; Voelker, D. R.; Feigenson, G. W., Membrane lipids: where they are and how they behave. Nat Rev Mol Cell Biol 2008, 9 (2), 112-24.
B58. Dupont, E.; Prochiantz, A.; Joliot, A., Penetratin Story: An Overview. Methods Mol Biol 2015, 1324, 29-37.
B59. Lonn, P.; Dowdy, S. F., Cationic PTD/CPP-mediated macromolecular delivery: charging into the cell. Expert Opin Drug Deliv 2015, 12 (10), 1627-36.
B60. Lonn, P.; Kacsinta, A. D.; Cui, X. S.; Hamil, A. S.; Kaulich, M.; Gogoi, K.; Dowdy, S. F., Enhancing Endosomal Escape for Intracellular Delivery of Macromolecular Biologic Therapeutics. Sci Rep 2016, 6, 32301.
B61. LaBelle, J. L.; Katz, S. G.; Bird, G. H.; Gavathiotis, E.; Stewart, M. L.; Lawrence, C.; Fisher, J. K.; Godes, M.; Pitter, K.; Kung, A. L., et al., A stapled BIM peptide overcomes apoptotic resistance in hematologic cancers. J Clin Invest 2012, 122 (6), 2018-31.
B62. Chanphai, P.; Bekale, L.; Tajmir-Riahi, H. A., Effect of hydrophobicity on protein-protein interactions. Eur Polym J 2015, 67, 224-231.
B63. Dyson, H. J.; Wright, P. E.; Scheraga, H. A., The role of hydrophobic interactions in initiation and propagation of protein folding. P Natl Acad Sci USA 2006, 103 (35), 13057-13061.
B64. Young, L.; Jernigan, R. L.; Covell, D. G., A Role for Surface Hydrophobicity in Protein-Protein Recognition. Protein Sci 1994, 3 (5), 717-729.
B65. Berndt, P.; Fields, G. B.; Tirrell, M., Synthetic Lipidation of Peptides and Amino-Acids—Monolayer Structure and Properties. J. Am. Chem. Soc 1995, 117 (37), 9515-9522.
B66. Chung, E. J.; Cheng, Y.; Morshed, R.; Nord, K.; Han, Y.; Wegscheid, M. L.; Auffinger, B.; Wainwright, D. A.; Lesniak, M. S.; Tirrell, M. V., Fibrin-binding, peptide amphiphile micelles for targeting glioblastoma. Biomaterials 2014, 35 (4), 1249-56.
B67. Trent, A.; Ulery, B. D.; Black, M. J.; Barrett, J. C.; Liang, S.; Kostenko, Y.; David, N. A.; Tirrell, M. V., Peptide amphiphile micelles self-adjuvant group A streptococcal vaccination. AAPS J 2015, 17 (2), 380-8.
B68. Black, M.; Trent, A.; Kostenko, Y.; Lee, J. S.; Olive, C.; Tirrell, M., Self-assembled peptide amphiphile micelles containing a cytotoxic T-cell epitope promote a protective immune response in vivo. Adv Mater 2012, 24 (28), 3845-9.

1. A peptide amphiphile comprising a hydrophobic tail and a bioactive peptide connected by an enzymatically-cleavable linker.

2. The peptide amphiphile of claim 1, wherein the enzymatically-cleavable linker is cathepsin-B (Cat-B) cleavable.

3. The peptide amphiphile of claim 1, wherein the bioactive peptide is a therapeutic peptide.

4. The peptide amphiphile of claim 3, wherein the therapeutic peptide binds to a protein within cells.

5. The peptide of amphiphile of claim 4, wherein the therapeutic peptide binds p53.

6. The peptide amphiphile of claim 5, wherein the therapeutic peptide comprises at least 70% sequence identity with SEQ ID NO: 1.

7. The peptide amphiphile of claim 1, wherein the hydrophobic segment comprises one or more alkyl chains.

8. A composition comprising a plurality of the peptide amphiphiles of claim 1 self-assembled into a nanostructure with the hydrophobic tails packed into a core of the nanostructure and the bioactive peptides displayed on the surface.

9. The composition of claim 8, wherein upon cleavage of the enzymatically-cleavable linkers, the bioactive peptides are released from the nanostructure.

10. The peptide amphiphile of claim 1, wherein the enzymatically-cleavable linker is flanked by detectably-distinct fluorophores.

11. The peptide amphiphile of claim 10, wherein the fluorophores form a FRET pair.

12. The peptide amphiphile of claim 11, wherein upon cleavage of the enzymatically-cleavable linker, a first fluorophore remains attached to the nanostructure and/or hydrophobic tail, and a second fluorophore remains attached to the bioactive peptide.

13. A composition comprising a plurality of the peptide amphiphiles of claim 10 self-assembled into a nanostructure with the hydrophobic tails packed into a core of the nanostructure and the bioactive peptides displayed on the surface.

14. The composition of claim 13, wherein upon cleavage of the enzymatically-cleavable linker, the functional peptide is released from the nanostructure and FRET between the fluorophores is diminished or eliminated.

15. A method of delivering a bioactive peptide to an in vivo location, comprising administering the peptide amphiphile of claim 1 to a cell, tissue, or subject.

16. The method of claim 15, wherein the peptide amphiphile or composition is monitored by fluorescence.

17. A method of delivering a bioactive peptide to an in vivo location, comprising administering the composition of claim 8 to a cell, tissue, or subject.

18. A method of delivering a bioactive peptide to an in vivo location, comprising administering the composition of claim 13 to a cell, tissue, or subject.
